(19) United States
(12) Patent Application Publication
(10) Pub. No.: US 2023/0210568 A1
(43) Pub. Date: Jul. 6, 2023

(54) BONE FIXATION SYSTEM AND ELEMENTS THEREOF

(71) Applicant: LIFESPANS LIMITED, Hong Kong (CN)

(72) Inventors: Christian Xinshuo FANG, Hong Kong (CN); Erica Ann BOLES, Hong Kong (CN); Man Chuen LEUNG, Hong Kong (CN); Sloan Austin KULPER, Hong Kong (CN); Ka Li Frankie LEUNG, Hong Kong (CN); Ahmed Abbas ALVI, Hong Kong (CN); Marilyn Janice OENTARYO, Hong Kong (CN); Mohd Rezaul Karim THARIM, Hong Kong (CN)

(73) Assignee: LIFESPANS LIMITED, Hing Kong (CN)

(21) Appl. No.: 17/924,012

(22) PCT Filed: May 8, 2021
(86) PCT No.: PCT/CN2021/092401

§ 371 Date: Nov. 8, 2022

(30) Foreign Application Priority Data
May 8, 2020 (HK) 32020007136.5

(51) Int. Cl. A61B 17/86; A61B 17/80

(52) U.S. Cl. CPC A61B 17/86; A61B 17/8057; A61B 2017/8655

(57) ABSTRACT

A fixation system (900) for the fixation of a first bone fragment (932) relative to a second bone fragment (934); the fixation system (900) comprising at least one first bone engagement element engagable with the first bone fragment (932), wherein the first bone engagement element has an elongate body portion, a proximal end, a distal end, and a passageway extending through the body portion from the proximal end to the distal end, at least one further bone engagement element engageable with the second bone fragment (934) and having a tensile element (920) securement portion for affixing a tensile element (920) to the further bone engagement element; wherein the tensile element (920) passing the first bone engagement element when engaged with the first bone fragment (932) is fixed relative to the further bone engagement element when engaged with the second bone fragment (934) by the tensile element (920) securement portion; such that the first bone fragment (932) is affixed relative to the second bone fragment (934) from tensile stress in the tensile element (920).

### TECHNICAL FIELD

The present invention relates to bone engagement devices and a system utilising such elements. More particularly, the present invention relates to a bone fixation system and fixation elements, and utilising such elements for fixation of bone tissue.

### BACKGROUND OF THE INVENTION

Within the field of orthopaedics and trauma, there exists the problem of aseptic loosening of fixation when fixing multi-fragmentary bone fractures, particularly those of the joint, wherein bone fragments must be secured in position to resist physiological loads from a multitude of directions.

At present, bone fixation implant technologies have relatively high rates of failure when used to repair multi-fragmentary fractures.

This is due in part to the loosen of connections between the implant components and patients' tissues (e.g. bone, muscle, ligament, and tendon) as these tissues are torn or slackened over time under load.

Sutures that are threaded directly into these tissues are particularly prone to such loss of tension, without which the sutures serve no structural role in securing bone fragments in place.

In the event of failure or loosening of a bone fixation system for reduction and fixation of adjacent bone fragments, such failure may result in non-union, incomplete union or incorrect union between bone fragment, and clinical and physiological complications resulting therefrom.

### OBJECT OF THE INVENTION

It is an object of the present invention to provide a bone fixation system and fixation elements thereof, which overcome or at least partly ameliorate at least some deficiencies as associated with the prior art.

### SUMMARY OF THE INVENTION

In a first aspect, the present invention provides a bone engagement element for engagement with bone of a subject and for releasable engagement with a tensile element, said bone engagement element having a longitudinal central axis and comprising body portion for engagement with bone tissue of a subject, said body portion having a distal end and a proximal end, wherein the body portion includes at least one engagement surface towards said proximal end and facing in a direction of at least from the distal end towards the proximal end; a bush member engageable with the proximal end of said body portion and having an aperture extending therethrough and being co-axial with said longitudinal central axis, said bush member having at least one complimentary engagement surface and facing in a direction of from at least the distal end towards the proximal end and said at least one complimentary engagement surface being complimentary to said at least one engagement surface of the body portion; and a fastener member threadedly engageable with the proximal end of said body portion, wherein the fastener member extends through the aperture of the bush member and being co-axial with said longitudinal central axis; wherein upon the fastener member being advanced in a direction of from the proximal end towards the distal end of the body portion, the fastener member urges the bush member in a direction of from the proximal end towards the distal end of the body portion; wherein upon the bush member being urged in a direction of from the proximal end towards the distal end of the body portion, the at least one complimentary engagement surface of the bush member is urged towards said at least one engagement surface of the body portion to lock any elongate element disposed between the at least one engagement surface and the at least one complimentary engagement surface.

The bone engagement element may be a bone screw. The bone screw may be a cortical bone screw or cancellous bone screw.

The bone engagement element may be a locking screw. The locking screw may be for engagement with a threaded aperture in an intermedullary fixation device.

The fastener member preferably includes a threaded portion for threaded engagement with the proximal portion of the body portion, and an engagement portion for receiving for receiving an external drive element to allow for rotation of the fastener member about its longitudinal axis.

The bush member is preferably axially movable relative to the longitudinal axis of the body portion, and wearing the bush member is restricted from rotational movement relative to the body portion about the longitudinal axis of the body portion.

The bush member and the body portion may be engaged by way of a key cooperate structure which prevents rotational motion of the bush member relative to the longitudinal axis of the body portion.

The engagement surface may be inclined radially outwardly and proximally so as to provide an outwardly extending inclined engagement surface. The complementary engagement surface may be distantly facing and inclined in a direction of outwardly radially and proximately.

The engagement surface and the complimentary engagement surface may be radially outwardly offset from the longitudinal central axis.

A recess may be provided at the proximal end portion of the body portion for receiving the bush member, and there is further provided one or more channels through which a tensile element may extend. The one or more channels channel may be provided as one or more passageways.

In a second aspect, the present invention provides fixation system for the fixation of a first bone fragment relative to a second bone fragment; said system comprising a plurality of bone engagement elements according to any one of the first aspect; and one or more tensile members for engagement with said plurality of bone engagement elements; wherein upon said tensile members engaged with said plurality of bone engagement elements and said bone engagement elements engaged with two or more bone fragments, said two or more bone fragments rigidly affixed relative to each other from tensile stress in said tensile member.

In a third aspect, the present invention provides a fixation system for the fixation of a first bone fragment relative to a second bone fragment; said system comprising:
at least one first bone engagement element engagable with a first bone fragment, wherein said bone engagement element has an elongate body portion, a proximal end, a distal end, and a passageway extending through the body portion from the proximal end to the distal end,
at least one further bone engagement element engageable with a further bone fragment and having a tensile element securement portion for affixing a tensile element to said at least one further bone engagement element;
wherein upon said tensile element passing the least one first bone engagement element when engaged with a first bone fragment is fixed relative to the at least one further bone engagement element when engaged with a first bone fragment by said tensile element securement portion; such that the first bone fragment is affixed relative to the further bone fragment from tensile stress in said tensile element material.

The least one first bone engagement element may include a tensile element securement portion for affixing said tensile element to said least one first bone engagement element at the proximal end.

The least one first bone engagement element may include a tensile element securement portion for affixing said tensile element to said least one first bone engagement element at the proximal end.

The least one first bone engagement element may include a tensile element securement portion for affixing said tensile element to said least one first bone engagement element at the proximal end and the distal end.

The at least one further bone engagement element may include a tensile element securement portion for affixing a tensile element to said least one first bone engagement element at the proximal end.

The at least one further bone engagement element may include a tensile element securement portion for affixing said tensile element to said least one first bone engagement element at the proximal end.

The at least one further bone engagement element may include a tensile element securement portion for affixing said tensile element to said least one first bone engagement element at the proximal end and the distal end.

The at least a first bone engagement element may be a bone screw, and the bone screw may be a bi-cortical bone screw.

The at least a further bone engagement element may be a bone screw, and the bone screw may be a bi-cortical bone screw.

The fixation system provides a three-dimensional structural support arrangement, which opposes relative movement between the first bone fragment and the further bone fragment.

In a fourth aspect, the present invention provides a method of securing a plurality of bone fragments relative to each other for fracture healing and union, said method includes the steps of

(i) engaging a plurality of bone engagement elements with such bone fragments, wherein said bone engagement element has an elongate body portion, a proximal end, a distal end, and a passageway extending through the body portion from the proximal end to the distal end and having a tensile element securement portion for affixing a tensile element thereto

(ii) affixing a tensile element to a first bone engagement element of plurality of bone engagement elements which is engaged with a first bone fragment of said plurality of bone fragments; and

(ii) tensioning said tensile element and engaging said tensile element with a further bone engagement element which is affixed to a further bone fragment of said plurality of bone fragments;
wherein upon tensile element passing the least one first bone engagement element when engaged with a first bone fragment is fixed relative to the at least one further bone engagement element when engaged with a first bone fragment by said tensile element securement portion; such that the first bone fragment is affixed relative to the further bone fragment from tensile stress in said tensile element material.

In a fifth aspect, the present invention provides a kit for securing a plurality of bone fragments relative to each other for fracture healing and union; said kit comprising plurality of bone engagement elements according to the first aspect; and one or more tensile elements.

### BRIEF DESCRIPTION OF THE DRAWINGS

In order that a more precise understanding of the above-recited invention can be obtained, a more particular description of the invention briefly described above will be rendered by reference to specific embodiments thereof that are illustrated in the appended drawings.

The drawings presented herein may not be drawn to scale and any reference to dimensions in the drawings or the following description is specific to the embodiments disclosed.

Any variations of these dimensions that will allow the subject invention to function for its intended purpose are considered to be within the scope of the subject invention.

Thus, understanding that these drawings depict only typical embodiments of the invention and are not therefore to be considered as limiting in scope, the invention will be described and explained with additional specificity and detail through the use of the accompanying drawings in which:

FIG. 1a demonstrates a three-part proximal humeral fracture;

FIG. 1b shows the displacement of the tuberosity fragment in a three-part humeral fracture illustrated in FIG. 1a;

FIG. 2 shows the three colour-coded fragments of the three-part fracture of the greater tuberosity and surgical neck as is demonstrated in FIG. 1a;

FIG. 3a shows an embodiment of the soft tissue retractor in a collapsed state according to the present invention;

FIG. 3b shows the soft tissue retractor shown of FIG. 3a in an expanded state according to the present invention;

FIG. 3c shows a photographic representation of a prototype model of an embodiment of the soft tissue retractor according to the present invention;

FIG. 4a shows an embodiment of the self-reaming nail according to the present invention;

FIG. 4b shows an enlarged view of the tip of the self-reaming nail as shown in FIG. 4a according to the present invention;

FIG. 4c shows a photographic representation of an embodiment of the self-reaming nail according to the present invention;

FIG. 4d shows a photographic representation of the tip of the self-reaming nail as shown in FIG. 4c according to the present invention;

FIG. 5a is a schematic representation of an embodiment of the suture tunnel screw according to the present invention;

FIG. 5b is a sectional view of the suture tunnel screw shown of FIG. 5a according to the present invention;

FIG. 6a shows the insertion of retrievers via a minimal incision during the suture retrieval process according to the present invention;

FIG. 6b shows the passing of multiple sutures through the retriever in one go during the suture retrieval process according to the present invention;

FIG. 6c shows the securing of the tuberosity fragments by retrieving the sutures during the suture retrieval process according to the present invention;

FIG. 6d shows the mounting of both ends of a first suture onto a T-lock deployment tool;

FIG. 6e illustrates the securing of sutures into a locking screw with the utilization of a T-lock deployment tool;

FIG. 7(a)(i) shows a side view of a first exemplary embodiment of a bone engagement element in an exploded view configuration;

FIG. 7(a)(ii) shows a front view of the embodiment of the bone engagement element of FIG. 7(a)(i) in an exploded view configuration;

FIG. 7(a)(iii) shows a perspective view of the embodiment of the bone engagement element of FIG. 7(a)(i) and FIG. 7(a)(ii) in an exploded view configuration;

FIG. 7(a)(iv) shows a sectional front view of the embodiment of the bone engagement element of FIG. 7(a)(i)-FIG. 7(a)(iii) in an exploded view configuration;

FIG. 7(a)(v) shows a sectional side view of the embodiment of the bone engagement element of FIG. 7(a)(i)-FIG. 7(a)(iv) in an exploded view configuration;

FIG. 7(b)(i) shows a side view of an exemplary embodiment of a bone engagement element in an assembled configuration;

FIG. 7(b)(ii) shows a front view of the embodiment of the bone engagement element of FIG. 7(a)(i)-FIG. 7(b)(i) in an assembled configuration;

FIG. 7(b)(iii) shows a perspective view of the embodiment of the bone engagement element of FIG. 7(a)(i)-FIG. 7(b)(ii) in an assembled configuration;

FIG. 7(b)(iv) shows a sectional front view of the embodiment of the bone engagement element of FIG. 7(a)(i)-FIG. 7(b)(iii) in an assembled configuration;

FIG. 7(b)(v) shows a sectional side view of the embodiment of the bone engagement element of FIG. 7(a)(i)-FIG. 7(b)(iv) in an assembled configuration;

FIG. 7(c)(i) shows a sectional front view of the embodiment of the bone engagement element of FIG. 7(a)(i)-FIG. 7(b)(v), with a tensile element extending therethrough, prior to locking of the tensile element to the bone engagement element;

FIG. 7(c)(ii) shows a sectional side view of the embodiment of the bone engagement element of FIG. 7(a)(i)-FIG. 7(c)(ii), with a tensile element extending therethrough, prior to locking of the tensile element to the bone engagement element;

FIG. 7(c)(iii) shows a sectional front view of the embodiment of the bone engagement element of FIG. 7(a)(i)-FIG. 7(c)(i), with a tensile element extending therethrough, after locking of the tensile element to the bone engagement element;

FIG. 7(c)(iv) shows a sectional side view of the embodiment of the bone engagement element of FIG. 7(a)(i)-FIG. 7(c)(iii), with a tensile element passing therethrough, after locking of the tensile element to the bone engagement element;

FIG. 7(d)(i) shows a schematic representation of a second exemplary embodiment of a bone engagement element according to the present invention in an exploded view configuration;

FIG. 7(d)(ii) shows a schematic representation of the bone engagement element as shown of FIG. 7(d)(i) in an assembled configuration;

FIG. 7(d)(iii) shows a photographic representation of the embodiment of FIG. 7(d)(i) and FIG. 7(d)(ii) of the bone engagement element of FIG. 7(d)(i) and FIG. 7(d)(ii);

FIGS. 8a to 8i show the steps of implementation of a bone engagement element according to the present invention;

FIG. 9 shows a schematic representation of an example of implementation of the present invention, depicting a tensioned network fixation system 900 provided by a plurality of bone engagement elements of the embodiment as depicted in FIGS. 7(a)(i) to 7(c)(ii).

FIG. 10a is a schematic representation showing a tensioned network of tensile elements provided as sutures which compresses bone fragment together in their anatomical position according to the present invention utilising the bone engagement element of FIG. 7(d)(i) to FIG. 7(d)(iii);

FIG. 10b is a three-dimensional model of FIG. 10a, which shows a tensioned network of the sutures compressing bone fragment together in their anatomical position;

FIG. 10c is a schematic representation showing the application of the tensioned network of suture shown of FIG. 10a and FIG. 10b in a proximal humeral fracture;

FIG. 11 shows the screw-nail locking mechanism according to the present invention utilising the bone engagement element of FIG. 7(d)(i) to FIG. 7(d)(iii);

FIG. 12a is a three-dimensional model of an embodiment of the Implant Device according to the present invention, for implantation in a three-part proximal humeral fracture model for simulation testing;

FIG. 12b is a three-dimensional model of the implant device of the Prior Art for implantation in a three-part proximal humeral fracture model for simulation testing;

FIG. 13a shows an anterior view of the simulation experiment setup of a three-part proximal humeral fracture model, with a rotation applied to the greater tuberosity fragment to simulate the muscle pulling force;

FIG. 13b shows a medial view of the simulation setup of the three-part proximal humeral fracture model as shown in FIG. 13a;

FIG. 13c shows a posterior view of the simulation setup of the three-part proximal humeral fracture model as shown in FIG. 13a;

FIG. 14a is a graphical representation of the force induced on the implant device of the present invention at the beginning of the simulation test as described in FIG. 13a, FIG. 13b and FIG. 13c;

FIG. 14b is a graphical representation of the force induced on the implant device of the present invention at the middle of the simulation test as described in FIG. 13a, FIG. 13b and FIG. 13c;

FIG. 14c is a graphical representation of the force induced on the implant device of the present invention at the end of the simulation test as described in FIG. 13a, FIG. 13b and FIG. 13c;

FIG. 15a is a graphical representation of the force induced on the implant device of FIG. 12b of the Prior Art at the beginning of the simulation test as described in FIG. 13a, FIG. 13b and FIG. 13c;

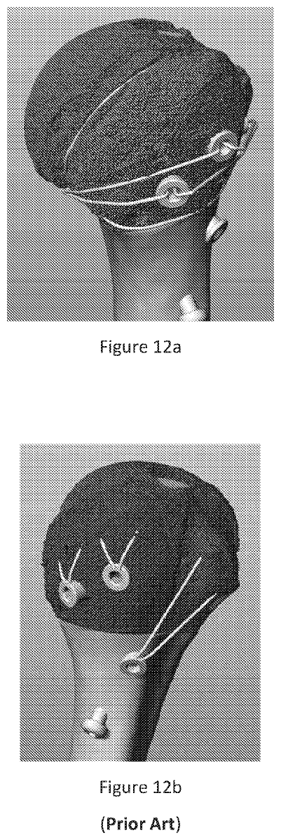

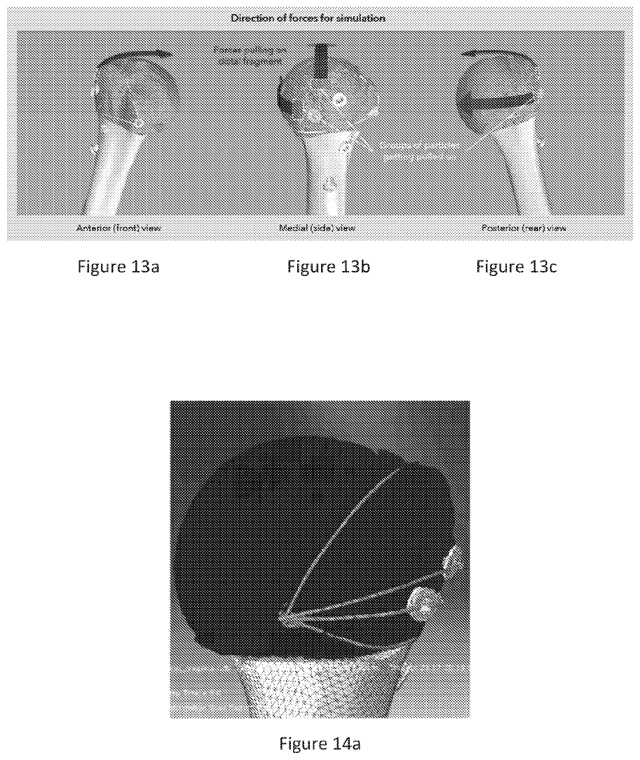

FIG. 13b is a graphical representation of the force induced on the implant device of FIG. 12b of the Prior Art at the middle of the simulation test as described in FIG. 13a, FIG. 16b and FIG. 16c;

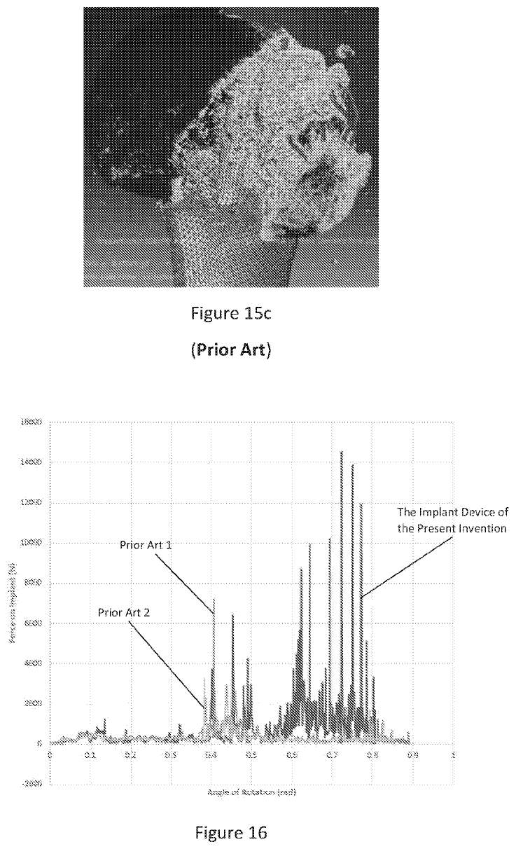

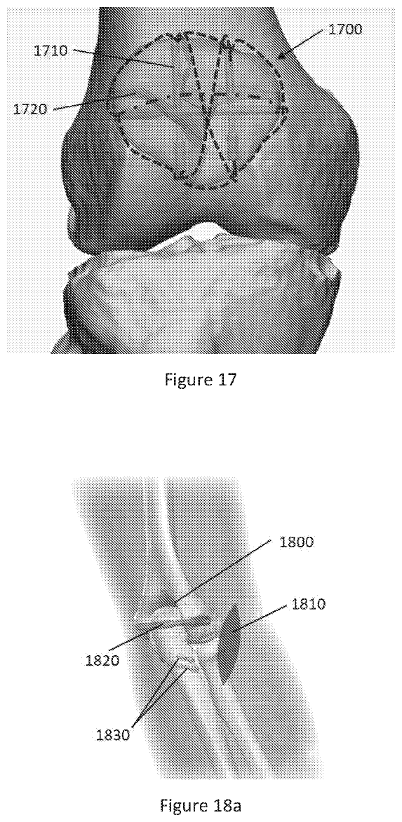

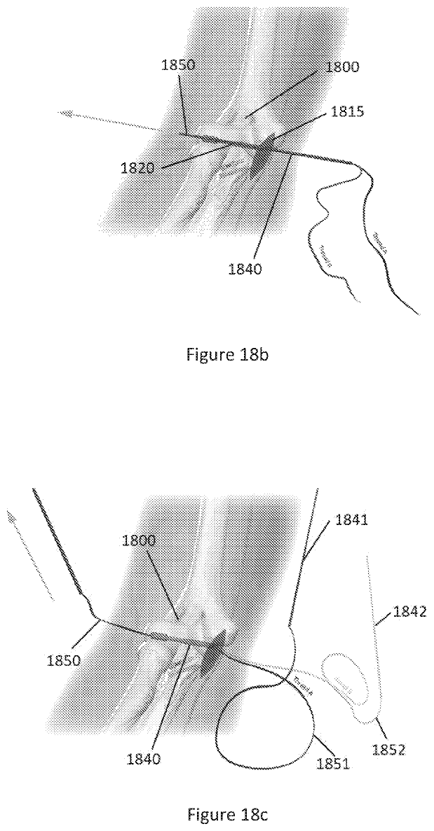

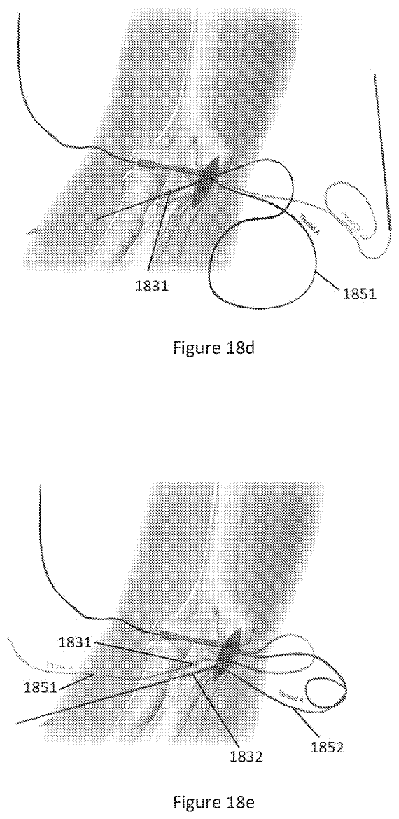

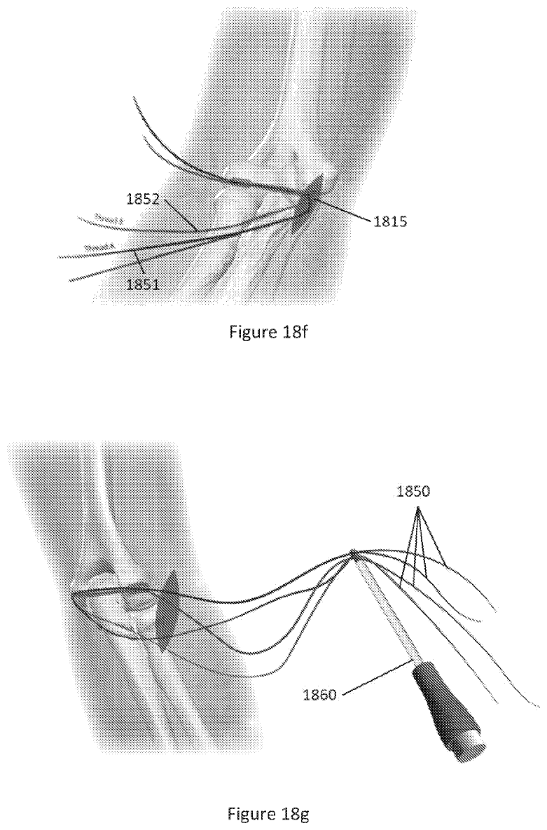

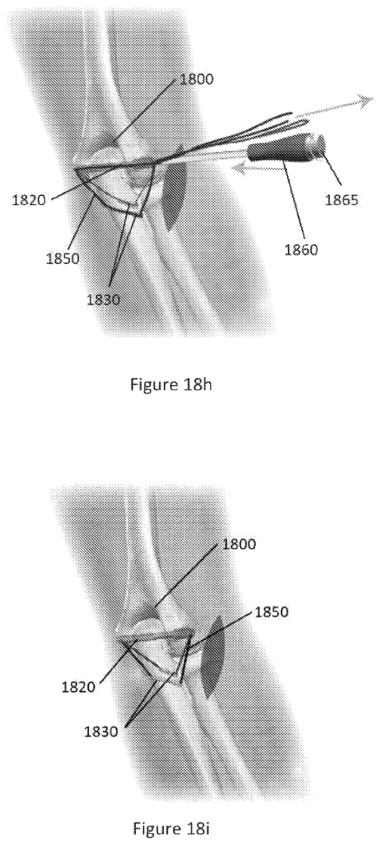

FIG. 15c is a graphical representation of the force induced on the implant device of FIG. 12b of the Prior Art at the end of the simulation test as described in FIG. 13a, FIG. 13b and FIG. 13c;

FIG. 16 is a graph of force on implant versus angle of rotation of the implant device of the present invention, in comparison with two other implant devices of the prior art;

FIG. 17 shows a schematic representation of a Patella Tensegrity Device consisting of T-lock screws and tensioned sutures to compress and fix the bone fragments together; and

FIG. 18a to FIG. 18i show the surgical technique to treat elbow instability by using the implant device according to the present invention.

### DETAILED DESCRIPTION OF THE DRAWINGS

The present inventors have identified shortcomings in bone fixation devices and systems of the prior art, and upon identification of the problems with the prior art, have provided a bone implant device which overcomes the problems of the prior art.

Observations of Prior Art by Present Inventors

Within the current art and generation of orthopaedic fixation implants, such devices may be considered to revolve around two main principles:
1) rigid components which resist bending torque and axial forces transmitted via bone with screws, plates and intramedullary nails being examples, and;
2) semi flexible or flexible components which resists tension through anchorage into soft tissue or by trapping of bone under a loop. With sutures and tension wire being typical examples.

The rigid components are generally dependent on force transmission into head tissue for stability, while flexible components resist tension.

There have been considerable engineering evolutions in the following phases:
(i) Compression plating—1970s,
(ii) Low contact biological plating—1990s,
(iii) Angle stable locking screws—2000s. (Uhthoff, Poitras et al. 2006)

In 2010s, most implant manufactures focused on anatomically pre-contoured implants and incorporation of variable angle locking screws—2010s.

These innovations have improved the ergonomics compromised the fixation strength. (Lenz, Wahl et al. 2015, Tank, Schneider et al. 2016)

Similarly, for intramedullary nails, the main evolutionary eras are incorporation of locking screws—1970s, use of reaming—1980s, use of titanium alloys and application to metaphyseal regions—2000s (Dilisio, Nowinski et al. 2016)

Suture material science and suture anchor technology mainly facilitated by popularization of arthroscopic surgery for sports and ligamentous reconstructions. Minimal invasive application of suture anchors—1990s, use of interference screws—1990s, use of bioabsorbable materials—2000s, use of endobutton in opposite cortex fixations—2000s, high strength polyethylene sutures—2000s, and knotless suture anchor systems—2010s. (Visscher, Jeffery et al. 2019)

The two areas of innovation are separate entities as trauma surgeons focused on rigid systems used for fracture fixation.

Sports and arthroscopic surgeons focused on tension systems soft tissue fixation.

The present inventors have observed and overlap between the two areas because of crossover between the sub-specialties.

Minimal invasive fracture fixation and arthroscopic assisted techniques are commonly preferred. Addressing the soft tissue component previously overlooked is now being increasingly addressed.

For fracture transition from hard tissue to soft tissue. locations under tension are attachments of muscle and important ligaments responsible for joint stability and function. Avulsion injuries at the bone-tendon or bone-ligament junctions are extremely common. They must be addressed together in comprehensive care of musculoskeletal injuries.

An example of such trauma is rotator cuff tuberosity avulsion fractures in proximal humerus fracture, epicondylar avulsions in elbow fracture subluxations, avulsions of trochanteric attachments in hip fractures, patella and quadriceps tendon avulsions in patella fractures, malleolar avulsion fractures in ankle injuries.

As noted by the present inventors, there does not exist an implant or implant or fixation system that address above identified problems. Commonly, additional use of suture anchors or rudimentary ‘docking’ techniques involving passing sutures or graft material drill holes as utilized with inferior mechanical results.

important factors for dictating strength of tensile implants—sutures and suture anchors are:
(i) firstly, the length/depth of the anchor fastened into bone material;
(ii) secondly the diameter of the suture material;
(iii) thirdly, the number of suture strands sharing the tension; and
(iv) fourthly, the quality of bone able to resist the tensile pull around the anchors. In essence, longer anchors, thicker sutures, more strands and strong bone improve pull-out resistance. (Visscher, Jeffery et al. 2019)

### REFERENCES

AO foundation “AO Surgery Reference.”
Bordoni, B., D. Lintonbon and B. Morabito (2018). “Meaning of the Solid and Liquid Fascia to Reconsider the Model of Biotensegrity.” Cureus 10(7): e2922-e2922.
Brais, G., J. Ménard, J. Mutch, G. Y. Laflamme, Y. Petit and D. M. Rouleau (2015). “Transosseous braided-tape and double-row fixations are better than tension band for avulsion-type greater tuberosity fractures.” Injury 46(6): 1007-1012.
Cho, N. S., S. C. Moon and Y. G. Rhee (2013). “Clinical Results of Various Surgical Techniques for Isolated Fracture of Greater Tuberosity of Humerus.” J Korean Fract Soc 26(2): 133-139.
Dilisio, M. F., R. J. Nowinski, A. M. Hatzidakis and E. V. Fehringer (2016). “Intramedullary nailing of the proximal humerus: evolution, technique, and results.” Journal of shoulder and elbow surgery 25(5): e130-e138.
Kralinger, F., M. Blauth, J. Goldhahn, K. Kach, C. Voigt, A. Platz and B. Hanson (2014). “The Influence of Local Bone Density on the Outcome of One Hundred and Fifty Proximal Humeral Fractures Treated with a Locking Plate.” J Bone Joint Surg Am 96(12): 1026-1032.
Lenz, M., D. Wahl, B. Gueorguiev, J. B. Jupiter and S. M. Perren (2015). “Concept of variable angle locking—evolution and mechanical evaluation of a recent technology.” Journal of Orthopaedic Research 33(7): 988-992.
Myers, T. W. (2020). “Tension-dependent structures in a stretch-activated system.” Journal of Bodywork and Movement Therapies 24(1): 131-133.
Naggar, L. (2018). “Surgical Management of Comminuted, Displaced Greater Tuberosity Fractures: A New Technique of Subacromial Spacer on Top of Double-Row Suture Anchor Fixation.” Joints 06(03): 211-214.
Palumbo, B., S. Gutierrez, B. Santoni and M. Mighell (2017). “Biomechanical Investigation of Locked Plate Fixation with Suture Augmentation in a Comminuted Three-Part Proximal Humerus Fracture Model.” Open Journal of Orthopedics 7: 180-191.
Shea, G. K., K. Hoi-Ting So, K. W. Tam, D. K. Yee, C. Fang and F. Leung (2019). “Comparing 3 Different Techniques of Patella Fracture Fixation and Their Complications.” Geriatr Orthop Surg Rehabil 10: 2151459319827143.
Tank, J. C., P. S. Schneider, E. Davis, M. Galpin, M. L. Prasarn, A. M. Choo, J. W. Munz, T. S. Achor, J. F. Kellam and J. L. Gary (2016). “Early mechanical failures of the synthes variable angle locking distal femur plate.” Journal of orthopaedic trauma 30(1): e7-e11.
Uhthoff, H. K., P. Poitras and D. S. Backman (2006). “Internal plate fixation of fractures: short history and recent developments.” Journal of orthopaedic science: official journal of the Japanese Orthopaedic Association 11(2): 118-126.
Visscher, L. E., C. Jeffery, T. Gilmour, L. Anderson and G. Couzens (2019). “The history of suture anchors in orthopaedic surgery.” Clinical Biomechanics 61: 70-78.

### Present Invention

In particular, the present invention is concerned with resisting the migration bone fragments from their fixed positions relative to each other due to tensile physiological loads such as those arising from ligaments and tendons.

If insufficiently resisted by an implant, such tensile physiological loads may lead to the separation of bone fragments and the consequent prevention of proper healing of bone fractures following surgery, and cause associated clinical and physiological problems and consequences.

The present inventors have noted that the two above-identified principal fixations are typically not used together simultaneously for fixation of bone fragments relative to each other, these being:
1) rigid components which resist bending torque and axial forces transmitted via bone with screws, plates and intramedullary nails being examples, and;
2) semi flexible or flexible components which resists tension through anchorage into soft tissue or by trapping of bone under a loop. With sutures and tension wire being typical examples.

Thus, the present inventors have provided an invention that seeks to address the clinical problem of aseptic loosening by providing novel means for surgeons to create and novel bone fixation system which implements “tensegrity structures” that are highly resistant to physiological loads of multiple directions and magnitudes.

In this case, “tensegrity structures” are composed of a combination of tensile elements (for example flexible suture, flexible cable, or other flexible and appropriate articles) and compressive elements for example a rigid screw, fastener, nail or the like, wherein the tensile members are used to connect between the ends of the compressive members.

It should be noted that such flexible components, in addition to supporting tensile loads, substantially to not support compressive loads and as such, articles such as suture, flexible cable, suitably flexible wires, are considered appropriate.

This is in contrast to the current state of the art and prior art of implant technology, that generally uses sutures to merely anchor tissues to screws or nails by threading them through bone/muscle/tendon.

The present inventors, upon identification of the problems and shortcomings of the prior art, throughout the development of the present invention, have sought to utilise concepts from non-related technical fields to the clinical human environment, such as civil engineering which is a field of endeavour from which technology is not applied within the field of orthopaedic engineering and surgery, in which the concept of tensegrity has been developed to design bridges and other structures that are highly-resistant to forces from multiple directions.

Accordingly, the present invention represents the application of the tensegrity concept being applied to orthopaedic biomedical engineering for the purpose of creating bone fixation implants with true tensile-compressive (“tensegrity”) structures.

Further, for the provision of such a system, the ability to tension the tensile elements, such as suture material, and to be able to adjust the tension of tensile elements extending between bone engagement elements, and a fix the tensile element relative to the bone engage element, to allow for adjust ability and re tensioning or loosening of the tensile element has been identified as being an important aspect in such a system as is provided by the present invention.

The present inventors have identified that a bone engagement element, for use in such a system, is required to provide for ease of fixation and loosening of the tensile element and with that affecting the tension of the tensile element, or other tensile elements in the system when adjustment is made.

As such, the present invention provides a bone engagement element which provides for ease locking and unlocking of a tensile element to the bone engagement element, without the use of knots, and provides for ease of Inter operative adjustment during surgical procedures and re tensioning or loosening of tensile elements when required Inter operatively. Furthermore, the present invention provides for knotless engagement and a bone fixation element, such as suture material, to a bone fixation element such as a bone screw.

Advantageously, such a device of the present invention, reduces wear and abrasion on tensile elements, such as suture material and as such comment provides improved and enhanced longevity and effectiveness clinically and when the tensile element is under load.

### Example 1—Proximal Humerus Tensegrity-Based Implant System

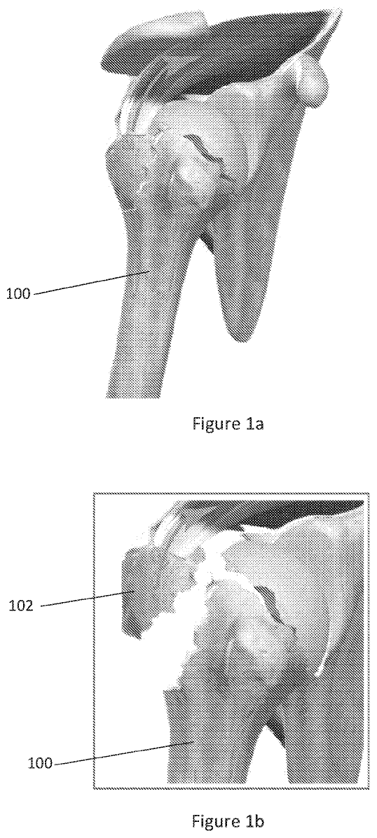

Referring to FIGS. 1a to 13c, there is shown an example of the present invention, and utilisation of fixation hardware element and system of the present invention, as well as surgical instruments in accordance with the present invention.

FIG. 1a demonstrates a three-part proximal humeral fracture of the humerus 100;

FIG. 1b shows the displacement of the tuberosity fragment 102 in a three-part humeral fracture illustrated in FIG. 1a;

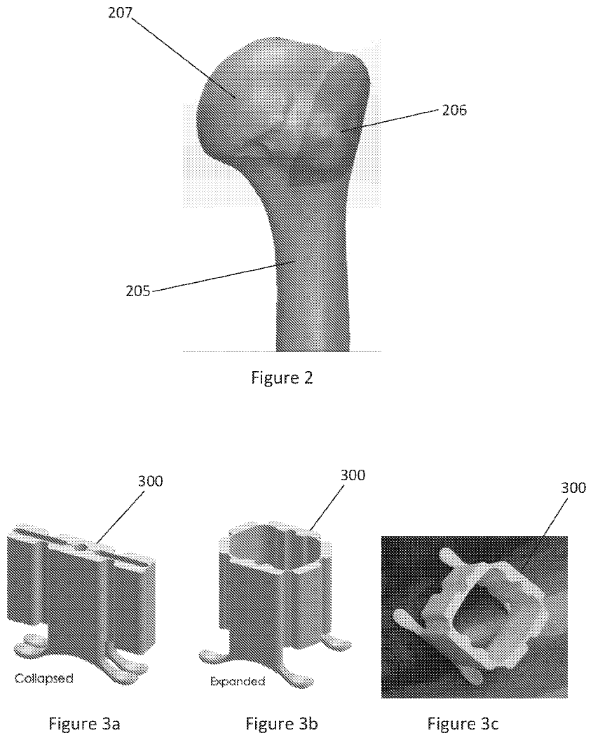

FIG. 2 shows the three fragments of the three-part fracture 105, 106, 107 of the greater tuberosity and surgical neck as is demonstrated in FIG. 1a;

Referring now FIGS. 3a, 3b and 3c there is shown an embodiment of the soft tissue retractor 300 in a collapsed state according to the present invention, whereby FIG. 3b shows the soft tissue retractor 300 shown of FIG. 3a in an expanded state according to the present invention, and FIG. 3c shows a photographic representation of a prototype model of an embodiment of the soft tissue retractor 300 according to the present invention;

The soft tissue retractor 300 is utilised to hold open soft tissue along its fibre orientation without tearing crosswise during implant insertion, and is collapsible for minimally invasive insertion/removal through the incision; easy-to-use and commercially economical.

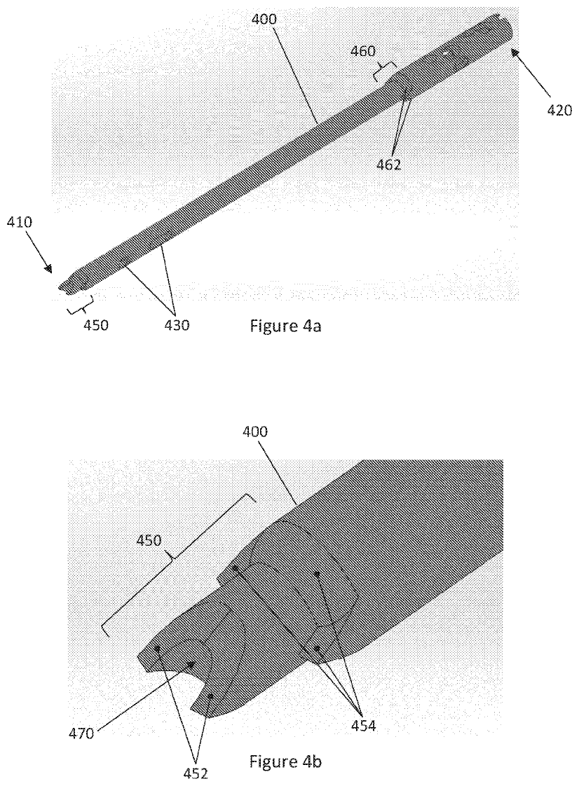

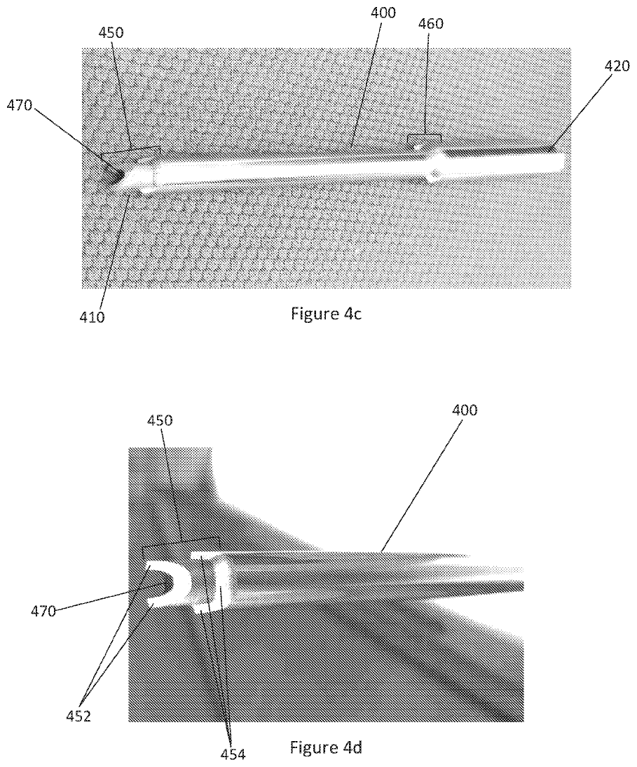

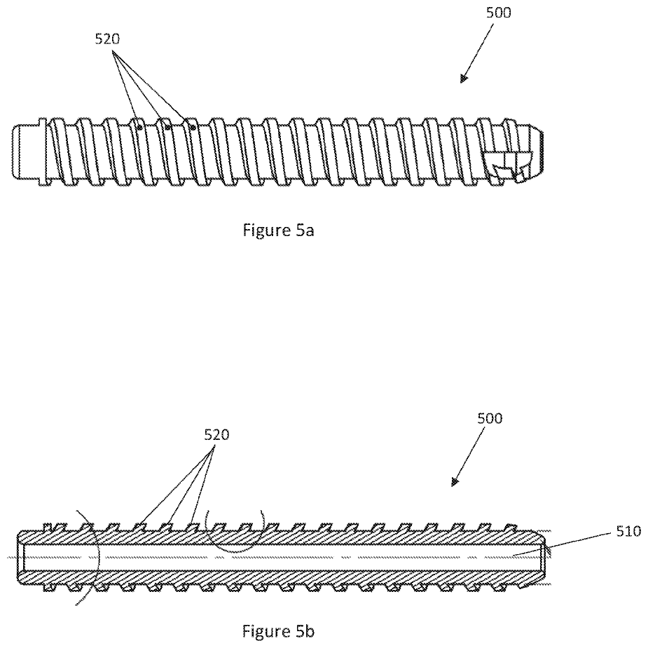

Referring to FIGS. 4a to 4d. there is shown an embodiment of the self-reaming nail 400 according to the present invention Self-reaming screws and nails 400 according to the present invention, advantageously provide for no additional reaming step or reaming instrument required to insert the screws or nails 400.

The self-reaming nail 400 has a distal end 410 and a proximal end 420, a set of apertures 430 towards the distal end 410 and a set of apertures for 404 towards the proximal end 420.

Located at the distal end 410, there is a cutting portion 450 comprised of a plurality of cutting surfaces or facets 452 and 454.

Furthermore, located towards the proximal end 420, there is a further cutting portion 460 comprising surfaces or facets 462.

There may also be provided a central passage 470, which extends through the nail 400 from the proximal end for 20 towards the distal end 410, and passes out of the nail 400 through cutting portion 450 so as to provide passage through the nail 400 in the form of a central lumen.

FIGS. 5a and 5b show a schematic representation of an embodiment of the suture tunnel screw 500 according to the present invention.

Suture tunnel screws 500 according to the present invention are cannulated screws 500 having a lumen 510 extending through, and a screw thread 520. such suture tunnel screws 500 to create a tunnel all the way through the bone making it possible to wrap sutures around hard-to-reach areas of the bone and create a web of tensioned sutures to anatomically compress and fix bone fragments in place, whereby the tunnel acts as a sheath to protect the bone by spreading stress at the suture.

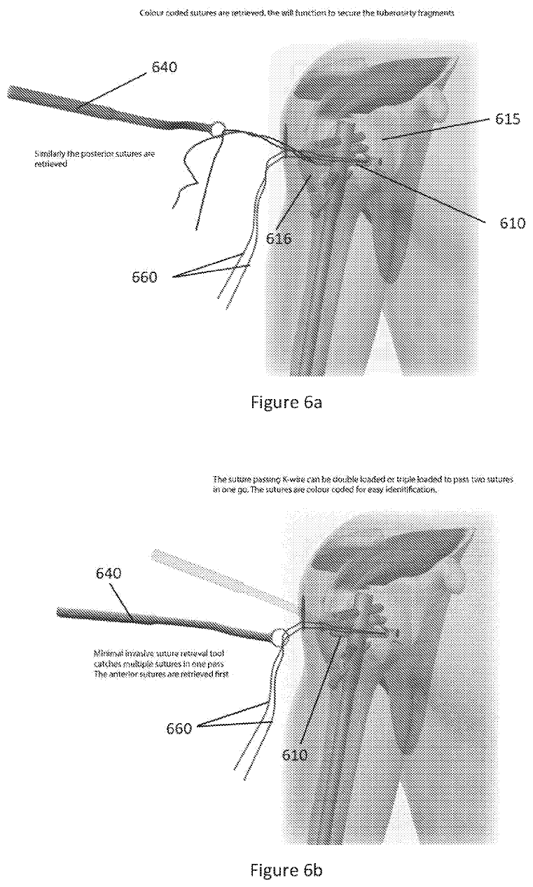

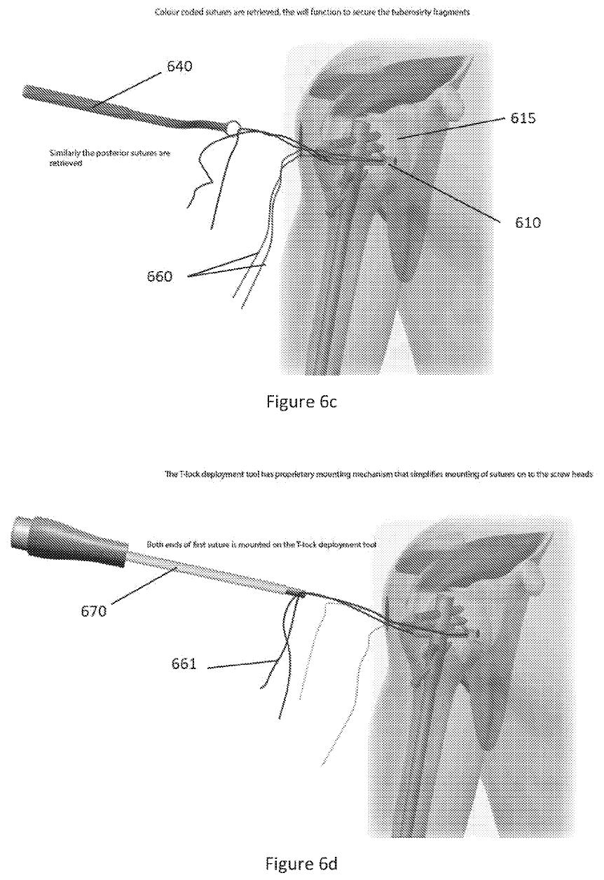

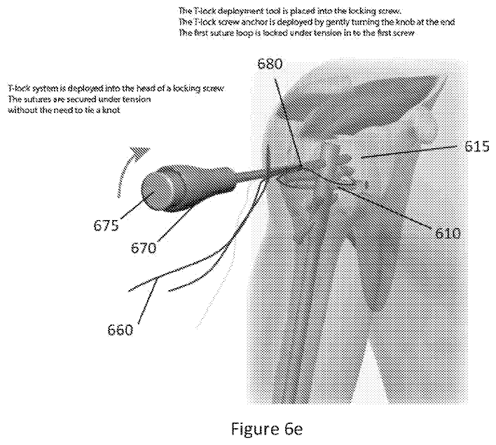

FIGS. 6a to 6e demonstrate the surgical process for treating a proximal humeral fracture with the utilization of the bone fixation system according to the present invention.

FIG. 6a shows the insertion of retrievers 640 via a minimal incision 620 at the shoulder of the patient during the suture retrieval process according to the present invention, whereby The Tensegrity implant is constructed minimally invasive against the humerus bone fractures 615, 616 by percutaneous tools.

As is shown in FIG. 6a, there exist two or more bone engagement elements 610, which in this case are bone screws, engageable with a first and second bone fragments 615, 616, wherein said bone screw 610 has a passageway extending through its body portion from its proximal end to the distal end.

Targeting device/retriever 640 is inserted via a minimal incision 620. Heavy suture is passed percutaneously via a threading K-wire 630 from anterior to posterior.

FIG. 6b shows the passing of multiple sutures 660 through the passageway of the bone engagement element 610 with the utilisation of the retriever 640 in one go during the suture retrieval process according to the present invention, whereby the suture passing K-wire can be double loaded or triple loaded to pass two sutures 660 in one go. The sutures 660 may be colour coded for easy identification in embodiments of the invention.

Minimal invasive suture retrieval tool 640 catches multiple sutures in one pass. The anterior sutures are retrieved first.

FIG. 6c shows the securing of the tuberosity fragments 615, 616 by retrieving the sutures 660 using a suture retrieval tool 640 during the suture retrieval process according to the present invention. Preferably, colour coded sutures are retrieved, this will function to secure the tuberosity fragments 615, 616. Similarly, the posterior sutures are retrieved.

FIG. 6d shows the mounting of both ends of a first suture 660 onto a T-lock deployment tool 670, whereby T-lock deployment tool 670 has proprietary mounting mechanism that simplifies mounting of sutures on to the screw head. Both ends of first suture 660 is mounted on the T-lock deployment tool.

FIG. 6e illustrates the securing of sutures 660 into a locking screw 680 with the utilization of a T-lock deployment tool 670, whereby the T-lock deployment tool is placed into the one of the bone engagement elements 610. There is deployed a knob 675 at the end of the T-lock deployment tool 670 such that by gently turning the knob 675, the sutures 660 can be tension adjusted and locked, loosened, and relocked as needed, and then the sutures can be affixed into a secured position with one of the bone engagement elements 610. The first suture loop 660 is locked under tension into the bone engagement elements 610. The sutures are secured under tension without the need to tie a knot.

By passing the sutures 660 through the passageways of the one or more bone engagement elements 610, the bone fragments 615 which are engaged with the bone engagement elements 610 are affixed and held against each other by the tensile stress in the suture material 660, and as such provide bone fixation to a proximal humeral fracture.

As can be seen as is demonstrated, the suture retrieval instrument: grabs sutures that are threaded through tunnel screws and gathers them for tensioning and locking into the T-lock screw heads bone interface and prevents sutures from cutting through poor quality bone.

The T-lock deployment instrument provide for loose suture ends are mounted to the T-lock deployment instrument, sutures are tensioned and then locked into the T-lock screw head.

The quick connect bone engagement or suture locking screw (“T-lock screw”), quick connect locking mechanism to securely lock tensioned sutures to the screw head to create a stable tensegrity web of sutures; extremely fast and easy compared to threading and tying suture knots by hand.

The tensegrity suture web in accordance with the present invention, which provides a tensioned network of sutures compresses bone fragments together in their anatomical position; especially effective for fragile bone fragments under high tensile forces (from the pull of attached muscles) that are difficult to fix using other methods (e.g., screws, pins, cerclage wire).

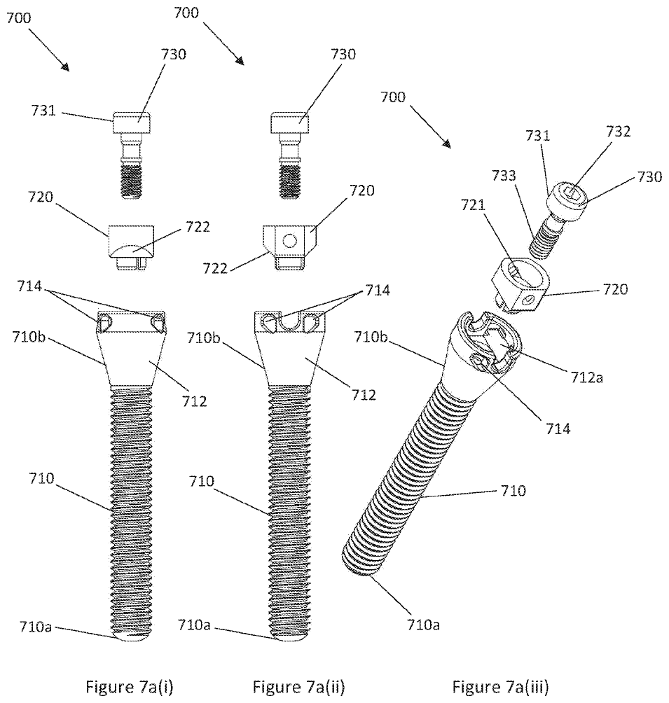

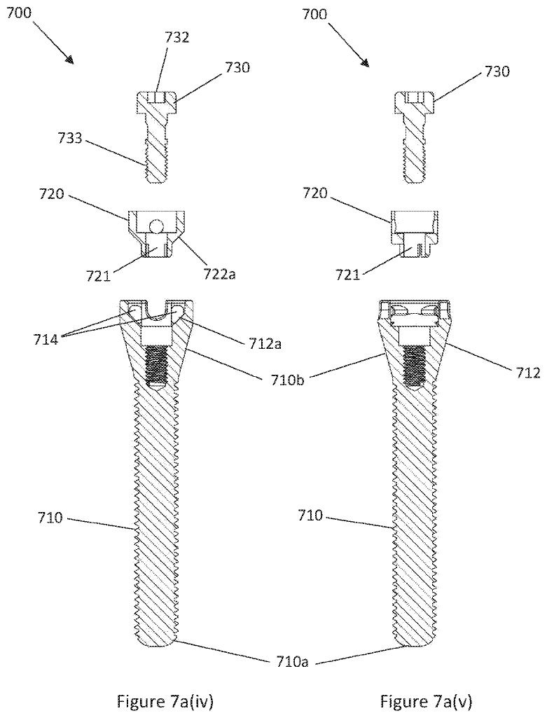

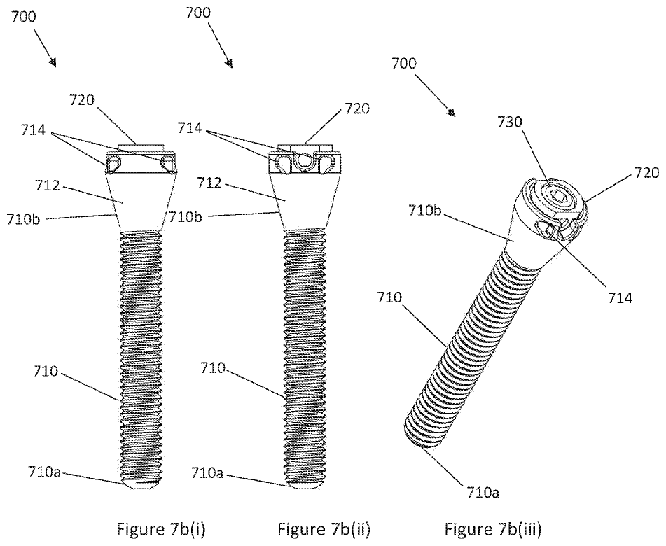

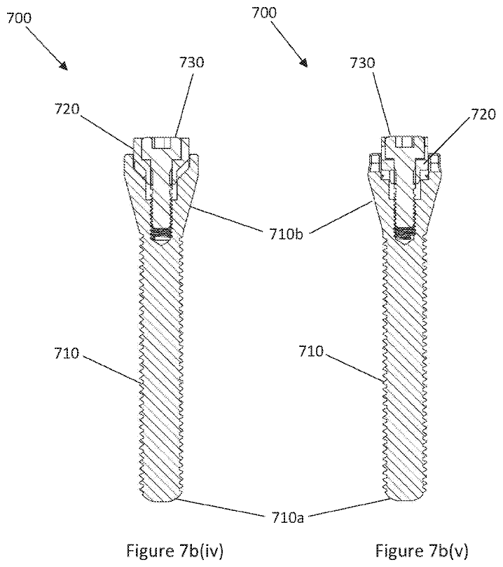

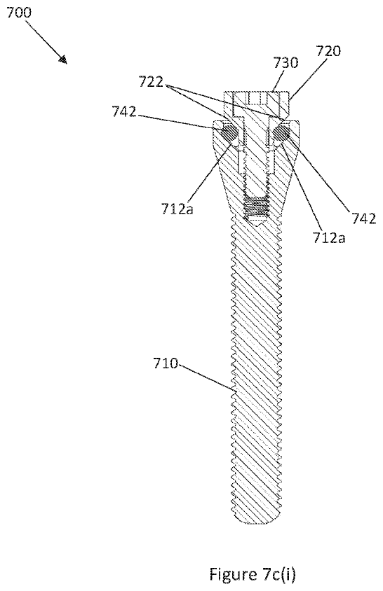

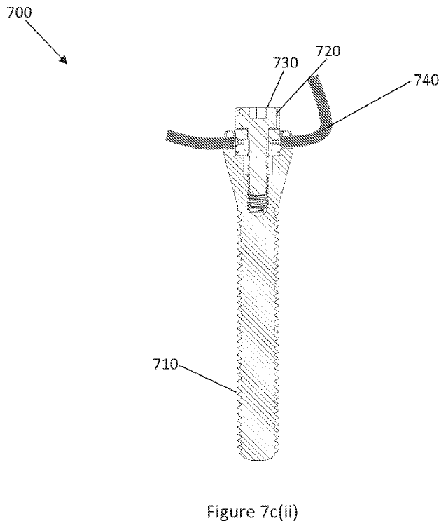

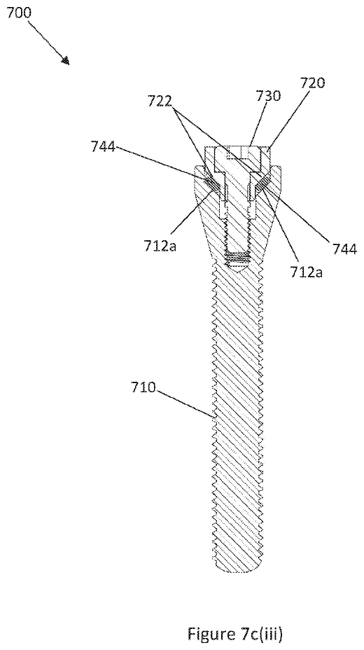

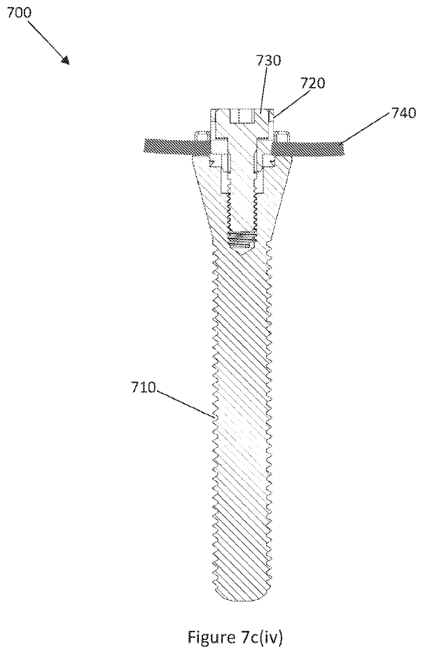

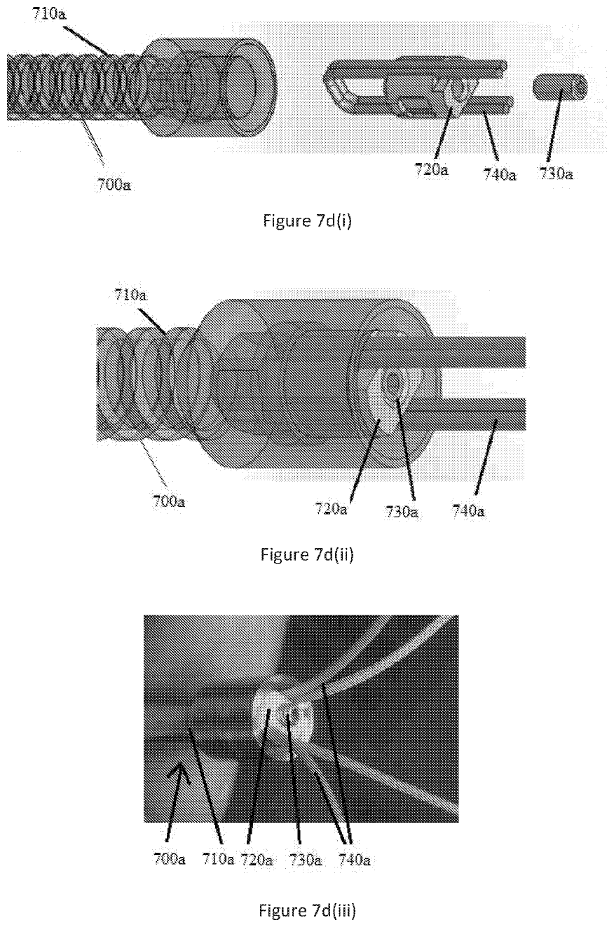

Referring to FIG. 7(a)(i) to FIG. 7(c)(ii) there is shown a bone engagement element 700 in accordance with the present invention.

The bone engagement element 700 includes a body portion 710 for engagement with bone tissue of a subject. The body portion 710 includes a distal end 710a and a proximal end 710b.

The bone engagement element 700 includes an engagement portion 710c, which in this case is depicted by a threaded region, which may be engaged with bone in the case of the device a screw such as a cortical bone screw, or engagement with another fixation device such as a locking element for engagement with a threaded aperture in an intermedullary device such as an intermedullary nail.

In the case that the bone engagement element is a bone screw, such as a cortical bone screw or a cancellous bone screw or a by cortical bone screw, the bone engagement element engages with the bone along the threaded section of the engagement portion 710c of the device.

In the case that the bone engagement element is a locking element such as for engagement with a threaded aperture in an intermedullary device, then the bone engagement element engages with the bone of a subject substantially at the proximal portion 712 of the bone engagement element which, in the present example, is depicted as a truncated conical head portion.

In any event, and as will be understood by those skilled in the art, regardless of the type of screw an engagement, both bone screws and locking screws of the type as described and as depicted, bone engagement elements within the art.

The bone engagement element 700 further includes a bush member 720 and a fastener member 730, whereby the fastener member 730 provides for engagement and locking and releasing of the bush member 720 relative to the proximal portion 712 of the body portion 710 of the bone engagement element 700.

The proximal portion 712 of the body portion 710 includes a recess for receiving the bush member 720, and the bush member has and aperture 721 extending through it, to allow the fastener member 732 extend through the bush member 720 and into the proximal portion 712 of the body portion 710.

The fastener member 730 includes a threaded portion 733 to allow for threaded engagement with the proximal portion 712 of the body portion, and whereby the fastener member 730 includes and engagement portion 733 for receiving for receiving an external drive element to allow for rotation of the fastener member 730 about its longitudinal axis, and thus allowing the fastener member to urge the bush member 720 against the proximal portion 712 all the body portion 710 of the fastener member 700.

The bush member 720 is arranged, such that it may not rotate about the longitudinal axis of the body portion 710, and as will be appreciated by those skilled, this may be provided in different manners, for example by way of a key-type mechanism which allows the bush member 720 only move axially with respect to the body portion 710, and not rotate how about the longitudinal axis.

Within the recess in the proximal portion 712 the body portion 710, there is provided an engagement surface 712, which in the present embodiment is proximally facing and is inclined radially outwardly and proximally so as to provide an outwardly extending inclined engagement surface.

Provided on the bush member 720, there is a complementary engagement surface 722, which is distantly facing and inclined in a direction of outwardly radially and proximately.

Further, provided at the proximal portion 712 of the body portion of 710 of the engagement element, channels 714 through which a tensile element, such as a suture, maybe placed. in the present embodiment, the channel 714 add depicted as closed passageways at the periphery of the proximal portion 712 of the body portion 710. However, in other or alternative embodiments, such channels need not necessarily be passageways, but may be open channels.

Referring now to FIG. 7(c)(i) and FIG. 7(c)(ii) specifically, there is shown a tensile element comment in this case, which is represented as element 740, which extends through the channels 714 and is disposed between engagement surface 712a and complementary engagement surface 722 of the body portion and the bush member respectively.

As is now shown in FIG. 7(c)(iii) and FIG. 7(c)(iv), upon the bush member 720 being urged towards the body portion 710 by fastener member 730, the tensile element 740 which is disposed through channels 714 and between the engagement surface 712a and complementary engagement surface 722, is releasably engaged with and locked relative to the proximal portion 712 of the body portion 710 of the bone engagement element 700.

As will be noted and understood by those skilled in the art, the bone engagement element 700 of the present invention, as described above with reference to the exemplary embodiment, allows for releasable engagement both a tensile member, such as a suture material, with the bone engagement element 700, to allow for tensioning, re tensioning, loosening and disengagement of the tensile member 740 with the bone engagement element 710 if and when required.

the present invention, by virtue of the engagement surface 712a and the complementary engagement surface 722, provides a uniform compressive force against the tensile element 740, upon the bush member being urged towards the proximal portion 712 of the body portion 710, without rotation of the bush member 720 relative to the body portion 710.

As such, and advantageously, ending comparison with some devices of the prior art, the present invention does not cause pinching and grinding of the tensile element, and prevents fraying and damage of the tensile member Inter operatively when I search in is required to adjust the tension of the tensile member bye releasably engaging the bush member 720 in relation to the body portion 71.

Furthermore, by providing and increased contact area by the complementary engagement surface is 712a and 722, this increases the contact area and thus reduces the loading and stress applied to the tensile element 740.

This provides significant advantages clinically coma as in the event of damage to a tensile element such as suture, this would require the replacement and possibly re threading and readjustment of the suture in relation to other bone engagement elements and as such, can cause complications, delay and increased anesthesia time Inter operatively,

As will be understood by those skilled in the art, the present invention provides significant advantages over the prior art, by allowing these of release engagement adjustment and re-tensioning and tensioning of a tensile element, without damage and compromising the tensile element 740.

The bone engagement element 700 of the present invention provides for releasable engagement with a tensile element, such as a suture material. accordingly, the present invention provides for knotless engagement with such a tensile element with the proximal portion 712 of the bone engagement element 700, and releasable engagement and adjustment of such a tensile element relative to the bone engagement element 700.

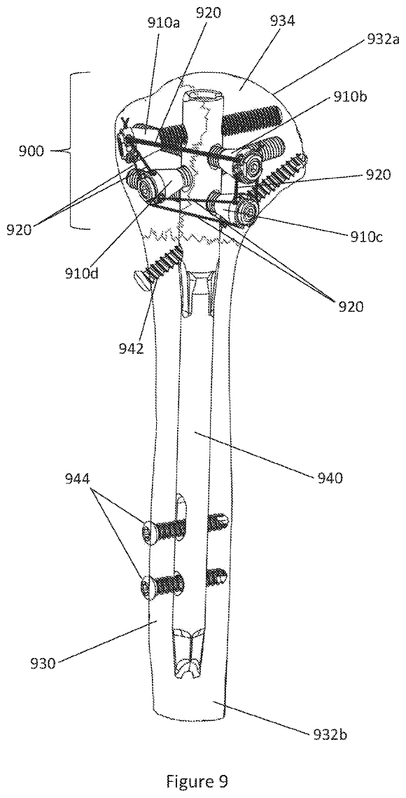

As such and as described in reference to FIG. 9 below, the present invention allows for adjustment, loosening, and re-tensioning tensile elements such as a suture extending between the proximal portions 712 of such bone engagement elements 700.

FIG. 7(d)(i) shows a schematic representation of a second exemplary embodiment of a bone engagement element 700a according to the present invention in an exploded view configuration; FIG. 7(d)(ii) shows a schematic representation of the bone engagement element 700a as shown of FIG. 7(d)(i) in an assembled configuration; and FIG. 7(d)(iii) shows a photographic representation of the embodiment of FIG. 7(d)(i) and FIG. 7(d)(ii) of the bone engagement element 700a of FIG. 7(d)(i) and FIG. 7(d)(ii).

The bone engagement element 700a and a comprises a body portion 710a, bush member 720a, fastener member 730a, which allows for the securement and locking of tensile element 740, similarly as described and shown in reference to the preceding embodiment.

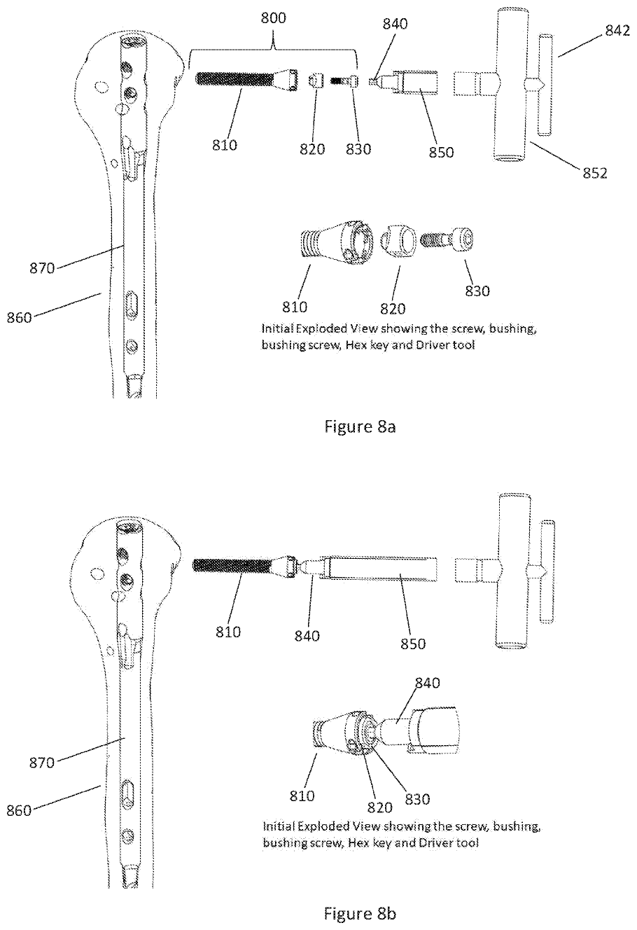

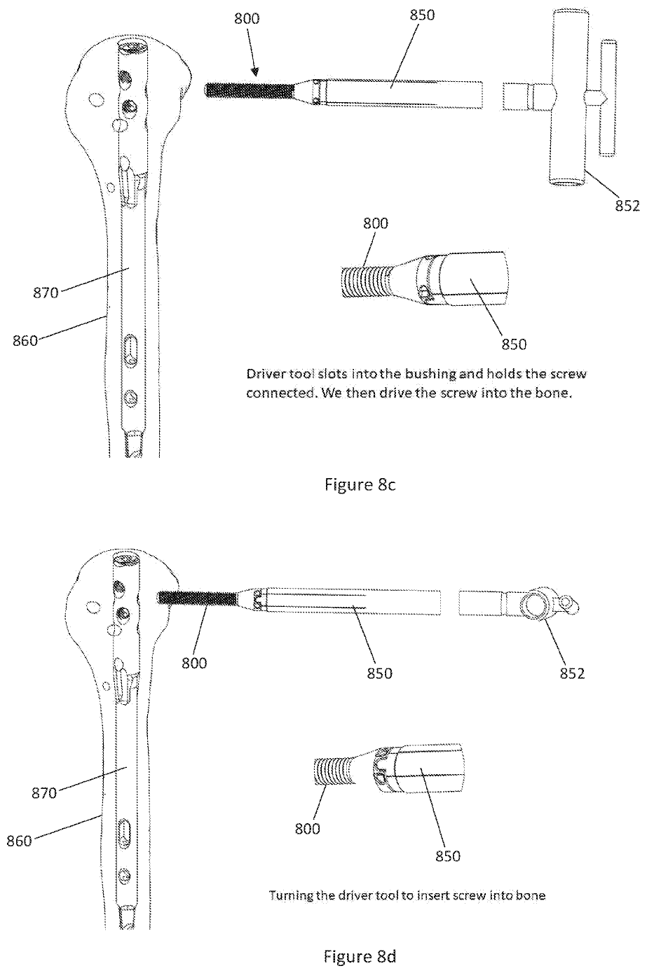

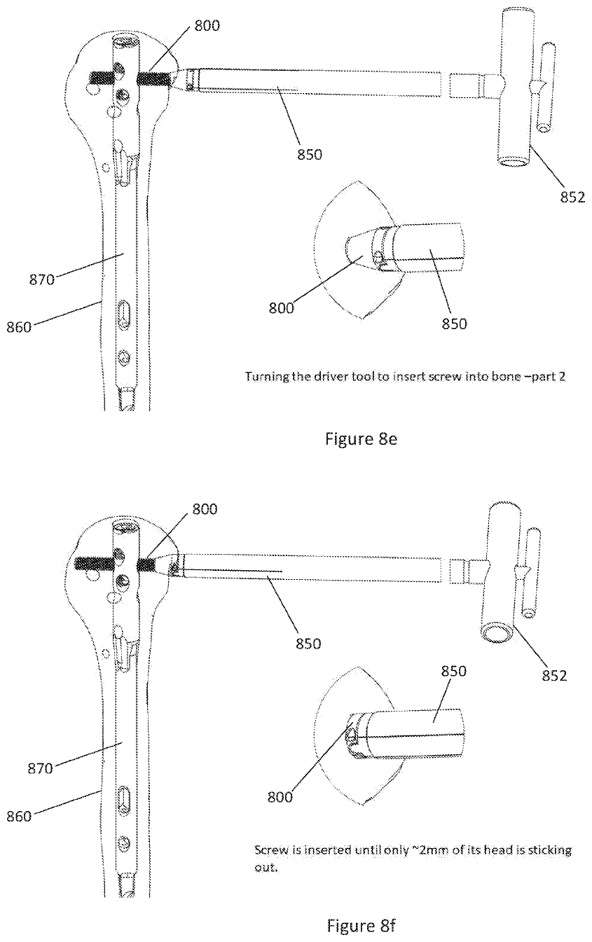

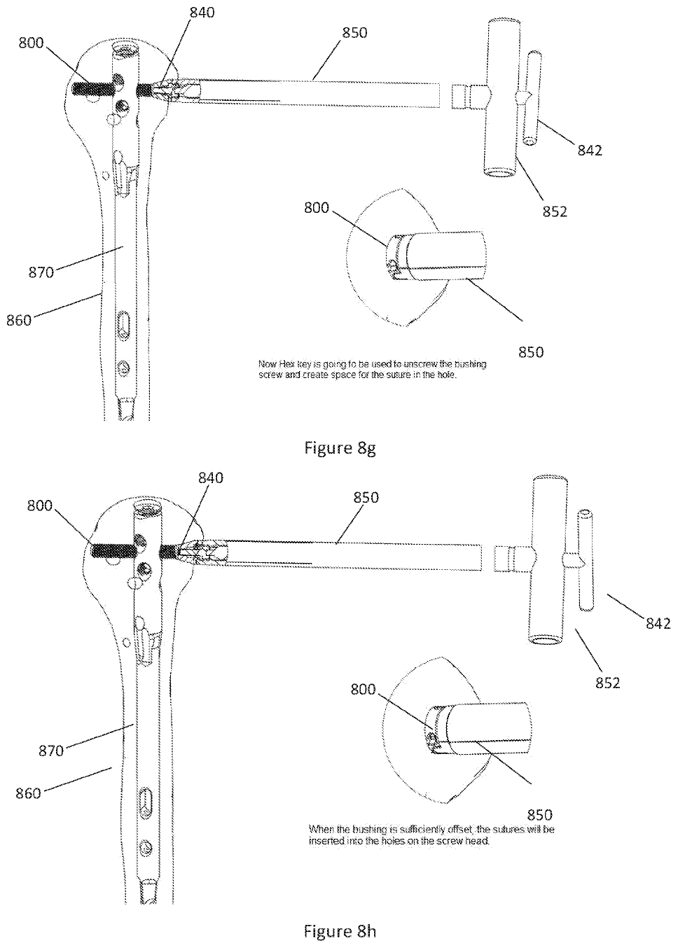

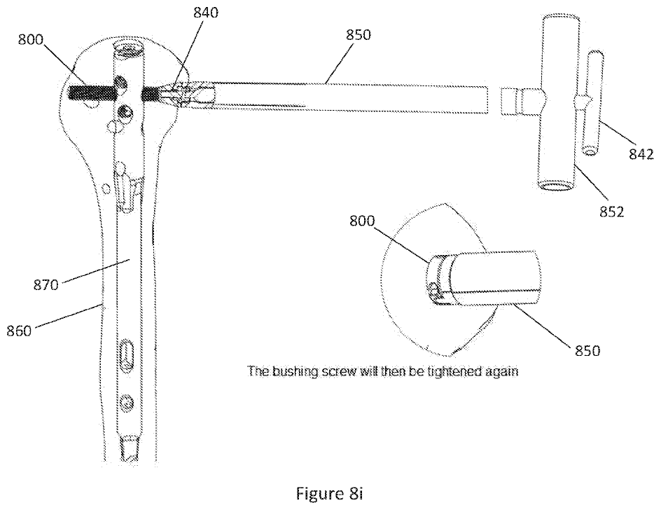

FIG. 8a to FIG. 8i depict the use of embodiment of a bone engagement element according to the present invention, such as that as based on FIGS. 7(a)(i) to 7(c)(ii), by way of example for engagement with an intermedullary nail 870 within a humerus bone 860.

Referring to FIGS. 8a to 8i, there is shown a bone engagement element 800 according to the present invention, which comprises a body portion 810, a bush member 820, and a fastener member 830.

There is also shown a fastener driver tool 840 end a main driver tool 850. The fastener driver tool 840 is located within a passage within the main driver tool 850, and is drivable by fastener driver tool handle 842. Main driver tool 850 is drivable independently from the faster driver tool by way of main driver tool handle 852.

As well as being independently rotatable about the central axis of each other, the faster driver tool 840 and the main driver tool 850 may be moved axially with respect to each other.

Referring now to FIG. 8a, there is shown the bone engagement element 800 in an exploded arrangement, as well as the faster driver tool 840 within the passage of the main driver tool 850.

In the present example, the fastener driver tool includes a hex-type of fitting at its distal tip, which is engagable with a complimentary hex recess in the head of the fastener 830.

Referring now to FIG. 8b, there is shown the bone engagement element 800 in unassembled form, and the fastener driver tool 840 engaged with the fastener member 830 and extending distally from the main driver tool 850.

As is now shown in FIG. 8c, the main driver tool 850 is moved towards the bone engagement element 800, and protrusions extending distally from the main driver tool 850 engage with slots in the proximal end of the bone engagement element 800, such that the main driver tool 850 is rotationally coupled to the bone engagement element 800.

Whilst in the present embodiment the main driver tool 850 is engageable with the proximal end of the bone engagement element 800 by way of a slotted mechanism, as will be understood and appreciated by those skilled in the art, other or alternative manners exist for such coupling which allows for ease of coupling an engagement between the main driver tool 850 and the bone engagement element, and the manner in which such detachable engagement there between is described and depicted in the present embodiment is non limiting, and any manner in which the main driver tool 850 may be engaged with the bone engagement element 800 and easily released, as will be understood by those skilled in the art, falls within the scope of the present invention.

Now as is shown in FIG. 8d, the main driver tool 850, by way of main driver tool handle 852, is used to urge the bone engagement element 800 into the bone 860 and towards the intermedullary nail 870 by handle 852, such that by turning main driver tool handle 852 of the main driver tool 850 the bone engagement element 800 is advanced towards the intermedullary nail 870. The fastener driver tool 840 rotates with the rotation of the main driver tool 850.

As will be understood, the detachable engagement between the main driver tool 850 and the bone engagement element 800 allows for ease of driving of the bone engagement element 800 into the bone 860. Again, any such engagement there between is equally as applicable in other or alternate embodiments.

As is shown in FIG. 8e, further turning of the main driver tool 850 advances the bone engagement element by further turning main driver tool handle 852 of the main driver tool 850, which in this embodiment is a locking element, into a threaded aperture in the intermedullary nail, and further turning of the handle 852 allows for the bone engagement element 800 to be advanced through the intermedullary nail 870.

As will be noted, at this point, the proximal end of the bone engagement element 800 has commenced penetration into the bone 860, and the distal end of the bone engagement element has protruded from the threaded aperture of the intermedullary nail 870, and extends further into bone material over the bone 860.

Referring to FIG. 8f, the bone engagement element 800 is urged into the bone 860 by further by turning main driver tool handle 852 of the main driver tool 850 until a requisite amount of the proximal end portion of the bone engagement element 800 is protruding from the bone, for example about 2 millimeters extending and protruding from the surface of the bone 860.

Depending on the particular clinical requirements, the surgeon shall advance the bone engagement element 800 to a sufficient depth within the bone 860 such that an applicable portion of the proximal end of the bone engagement element 800 is protruding from the bone 860.

As will be understood by those skilled in the art, the proximal end of the bone engagement element 800 protrudes sufficiently such that the channels or passageways through which the tensile element, such as a suture is passed by the surgeon, sufficiently protrudes from the cortex or outer surface of the bone 860 to allow for the threading of the tensile element therethrough.

Referring to FIG. 8g, following advancement of the bone engagement element 800 into the bone 860 to a requisite depth as described in reference to FIG. 8f, the fastener member of the bone engagement element 800 is loosened so as to allow the bush member to move in a proximal direction, the advancement of a tensile element, such as a suture material, through the passageways in the proximal end of the bone engagement element 800, similarly as described in reference to the preceding figures is then performed by a surgeon.

In order to do this, the requisite depth of the bone engagement element 800 is maintained in the bone by the surgeon holding the main driver tool handle 852 of the main driver tool 850 in its current position, so that the bone engagement element 800 is not rotated out of the bone, and at the same time turning the fastener driver tool 840 by way of the fastener driver tool handle 842 in the opposite direction as to when used for advancing the bone engagement element 800 into the bone and relative to the main driver tool 850.

Referring to FIG. 8h, as is shown, once the bush member has been released sufficiently in the proximal direction, the tensile elements may be inserted into the passageways of the bone engagement element, in accordance with the invention. as can be seen, the fastener driver tool 840 has been rotated relative to the main driver tool 850 which has been maintained at the requisite position so as to maintain the bone engagement element 800 at the correct depth of insertion within the bone 860.

Referring now to FIG. 8i, once the tensile element has been threaded through the passageways in the bone engagement element 800, the fastener driver tool 840 is then rotated in the opposite direction to the loosening direction a described in reference to FIG. 8g, whilst the main driver tool 850 is maintained at the present requisite position, which urges the bush member in a distal direction such that the tensile element is clamped between the complementary engagement services of the bone engagement element 800, in a manner which is described above and in accordance with the present invention.

Subsequently, the main driver tool 850 and the fastener driver tool 840 disengaged from the bone engagement element, and as will be understood, a further bone engagement element 800 is implanted within the bone, and the tensile element then advanced through the passageway of that further bone engagement element at a requisite tension, and then the bush member of that further bone engagement element is utilized to lock the tensile element at said requested tension.

As will be understood by those skilled in the art, throughout the process, it may be necessary to loosen and re-adjust or retention certain tensile elements, such that the bone fragments of a fracture are secured and appropriately maintained in the suitable positions and pressures against each other comma so as to promote and allow for bone healing and union,

As will be understood by those skilled in the art, the present invention provides a versatile system for the adjustment of tensile elements extending between bone engagement elements, thus providing a secure and stable 3-dimensional structure for the maintaining of bone fragments in requisite relative relationships to each other, whilst providing ease of adjustment intraoperatively by a surgeon or a clinician, and importantly, for such tensile elements such as suture material, does not damage or fray or corrupt the integrity of such tensile elements and thus, provides a secure engagement and structure to maintain the bone fragments in appropriate position throughout the bone healing and union process

Referring to FIG. 9, there is shown a schematic representation of an example of implementation of the present invention, depicting a tensioned network fixation system 900 provided by a plurality of bone engagement elements 910a, 910b, 910c, 910d of the type of the embodiment as depicted in FIGS. 7(a)(i) to 7(c)(ii).and a plurality of tensile elements 920 provided by suture material, which extend between the bone engagement elements 910.

In the present example, the bone 930 of a subject is a humerus bone with two fractures in the humeral head region of the bone, causing a first bone fragment 932 and a further bone fragment 934.

As is known by those skilled in the art, it is necessary to reduce the fractures and provide fixation of the bone fragments relative to each other to allow bone union and fracture healing.

In the present example, bone fixation is provided by use of an intermedullary nail 940, which extends through the intermedullary canal of the bone 930, and as is known by those skilled in the art can be used or fracture fixation.

The intermedullary nail 940 is inserted from the proximal end 932a towards the distal end 932b of the bone 930, and may be affixed to the bone 930 via bone screw 942 proximally and bone screws 944 distally.

In the present embodiment, the bone engagement elements 910a, 910b, 910c, 910d are locking screws or locking elements, which extend through threaded apertures at the proximal end of the intermedullary nail 940.

However, and as will be understood by those skilled in the art, in other end alternate embodiments, the bone engagement elements 910a, 910b, 910c, 910d maybe bone screws, such as cortical screws, fixation screws, by—cortical screws, typical trauma type fixation screws, screws for use in combination with fracture reduction plates and the like. As such, as will be understood, the bone engagement elements maybe any type of bone engagement element, and no limitation on such type of device applies to the present invention.

In the present example, the bone engagement elements 910a, 910b, 910c, 910d are the examples of bone engagement elements of FIGS. 7(a)(i) to 7(c)(ii) and hold the bone fragments, 932, 934 in relation to each other to provide for general reduction and maintaining the bone fragments in position for fracture union.

The tensile elements 920, which is suture material in the present example, passes through and is engaged with the bone engagement elements 910a, 910b, 910c, 910d such that such that the first bone fragment 932 is affixed relative to the further bone fragment 934 from tensile stress in said suture material.

Thus, this compresses the bone fragments together in their anatomical position, to assist in fracture union and healing.

In accordance with the present invention, the bone engagement elements, as described also above in reference to the present invention, allow for the ease of adjustment of the tension in the tensile elements inter-operatively, which has been observed by the present inventors as being a very important attribute an aspect from a clinical standpoint when repairing such fractures and utilizing such techniques for turn fracture reduction and securement appropriately of bone fragments relative to each other in a manner such that appropriate fracture union is achieved and biomechanical functionality restored to the subject in need of such treatment of bone fracture.

Again, such bone engagement elements need not necessarily be locking elements as in the presently depicted implementation of the invention, but maybe other types of bone screws such as cortical bone screws, trauma screws, but cortical screws and the like, as well as other nation hardware which may allow for engagement with the bone and the adjustable fixation and engagement of a tensile element, such as suture material, there with.

Furthermore, a mixture of different types of bone engagement elements may be also utilized in accordance with the present invention, and depending on the surgical and clinical requirements on a case by case basis, this determines the manner in which a surgeon or clinician may configure the bone reduction, where the bone engagement elements are affixed to the bone of the subject, as well as the orientation, direction and location of the tensile elements extending between debone engagement elements.

as we will also be understood, the bone engagement elements according to the present invention provide for knotless engagement of tensile elements, such as suture material, with the head portion of a bone engagement device such as a bone screw and as such, again, provides for ease of loosening, adjustment, tensioning or re tensioning and positioning of suture material Inter operatively, which is critical from a clinical standpoint such that a surgeon may appropriately adjust the tension in each case such that the bone fragments of the subject are appropriately positioned relative to each other as well as tensioned towards each other.

The three dimensional structure caused by the system of a plurality of bone engagement elements and tensile elements essentially provides a tensegrity-type of structure, and by virtue of the tensile elements or being intention, provides an inherently stable and strong three dimensional construct which affix is bone fragments relative to each other in three dimensions, thus resisting movement from anatomical loading during the fracture union and healing process.

As such, the present invention provides improved fixation end bone healing off bone fractures, in particular multiple bone fragment fractures, and provides a surgeon or clinician with the flexibility to provide an appropriate structure so as to maintain the integrity of the bone functionality by mechanically, as well as promoting appropriate and effective bone union and fracture healing.

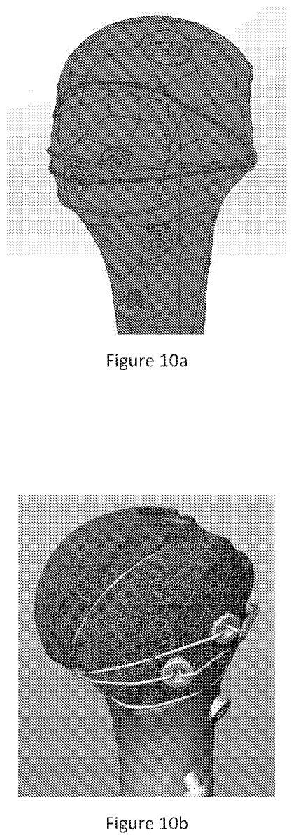

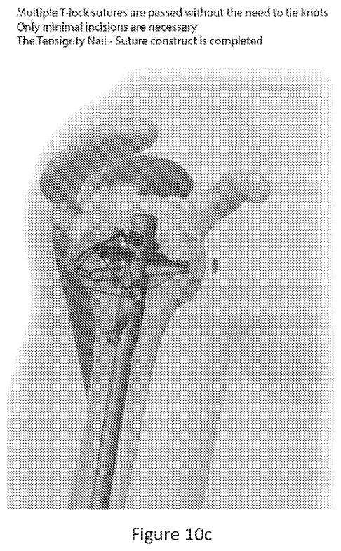

FIG. 10a is a schematic representation showing a tensioned network of sutures which compresses bone fragment together in their anatomical position, FIG. 10b is a three-dimensional model of FIG. 10a, which shows a tensioned network of sutures compressing bone fragment together in their anatomical position, and FIG. 10c is a schematic representation showing the application of the tensioned network of suture shown of FIG. 10a and FIG. 10b in a proximal humeral fracture.

FIG. 10b shows a three-dimensional model of FIG. 10a, which shows a tensioned network of sutures compressing bone fragment together in their anatomical position, and FIG. 10c is a schematic representation showing the application of the tensioned network of suture shown of FIG. 10a and FIG. 10b in a proximal humeral fracture.

As is shown in FIG. 10c, Multiple T-lock sutures are passed without the need to tie knots. Only minimal incisions are necessary.

As is shown, Screw—nail locking mechanism, and end cap engages locking of all screws going through the nail to prevent screws from toggling or backing out; implant acts as single stabile construct improving anchorage in bone:
(1) tighten and lock nail end cap, and
(2) the end cap engages the internal locking mechanism between each screw and the nail to create a single stable construct; prevents screws from backing out of the nail.

As is shown in FIG. 10c, Multiple T-lock sutures are passed without the need to tie knots. Only minimal incisions are necessary.

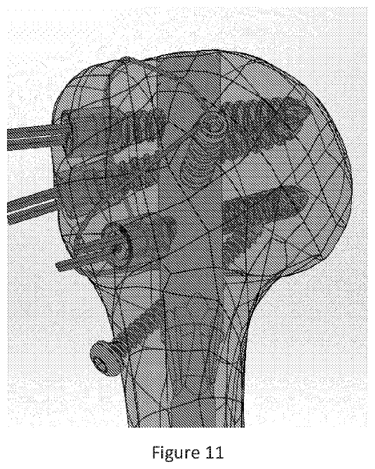

Referring to FIG. 11, the process includes:
(i) tightening and locking nail end cap
(ii) end cap engages the internal locking mechanism between each screw and the nail to create a single stable construct; prevents screws from backing out of the nail.

### 1.1 Experimental Results

Objectives: To compare the ability of the Proximal Humerus Tensegrity Device v1.0 vs. the conventional device to fix the greater tuberosity fragment undergoing a rotational pull in a 3-part proximal humeral fracture model using realistic virtual simulation testing. The force on the implant during rotation of the greater tuberosity fragment and the mode of failure was compared between the implant groups.

Hypothesis: The Proximal Humerus Tensegrity Device requires a higher load to failure and results in less displacement of the bone fragments compared to the conventional device of a nailing system with sutures of the prior art.

Materials:
Simulation software developed for realistic virtual testing of orthopaedic implants in bone;
Realistic 3D particle model of a CT scanned proximal humerus;
3D model of the Proximal Humerus Tensegrity Device v1.0 as shown in FIG. 12a; and
3D model of the conventional of a nailing system with suture loops according to the Prior Art as shown in FIG. 12b.

Protocol:
1. A CT scan of a human proximal humerus was converted to a realistic 3D particle model.
2. The fracture lines and fragments were assigned in the bone particle model to create a 3-part fracture of the greater tuberosity and surgical neck.
3. 3D models of the Proximal Humerus Tensegrity Device v1.0 and the conventional device were created, and each device was “implanted” in the bone model and the position of the nail, screws, and sutures was checked for correct positioning.
4. The material properties of the bone and implant materials were defined in the simulation software.
5. The rotational motion of the greater tuberosity fragment was defined in the software to simulate the direction of muscle pull on the bone fragment (FIG. 13a, 13b, 13c).
6. The simulation experiment was executed in Abaqus and the force on the implant during rotation of the greater tuberosity fragment was recorded and the mode of failure was observed.

Results:

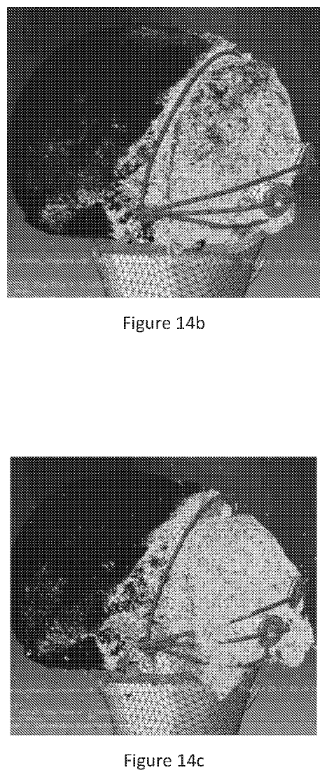

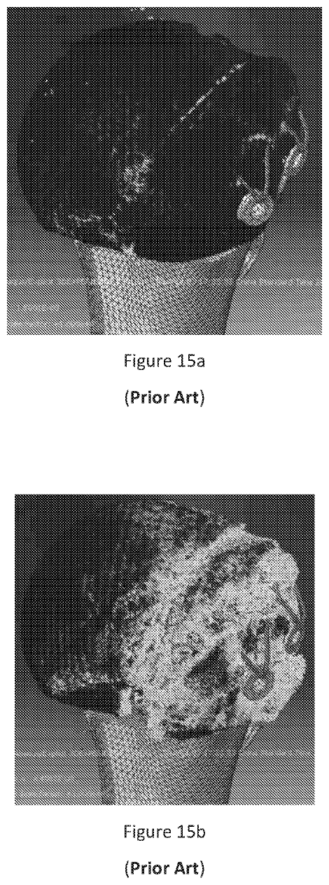

Snapshots taken during the simulation tests demonstrate that the Proximal Humerus Tensegrity Device (FIGS. 14a, 14b, 14c) and its tensegrity web of sutures was more effective at compressing and keeping the bone fragments together when compared to the conventional device of the Prior Art with suture loops (Figures, 15a, 15b 15c).

When the 3-part fracture was fixed with the Proximal Humerus Tensegrity Device v1.0, the force on the implant greatly increased in the later stages of fragment rotation (indicating prolonged fragment stability) and reached a maximum force up to two times higher than the force on the conventional device tested with two different suture configurations; in addition, the greater tuberosity fragment remained more intact for the Proximal Humerus Tensegrity Device v1.0 vs. the conventional device groups (FIG. 12b—Prior Art).

FIG. 16 is a graph of force on implant versus angle of rotation of the implant device of the present invention, in comparison with two other implant devices of the Prior Art, which demonstrates such an increase in force.

These results support the present inventors' hypothesis that a Tensegrity-Based Implant System is more effective at stabilizing fragile bone fragments in their anatomic position even under multidirectional loading and high muscle pulling forces when compared to the conventional devices currently on the market.

### Example 2: Patella Tensegrity-Based Implant System

To address these shortcomings of the current surgical techniques, the present invention provides a Patella Tensegrity Device incorporating features of our Tensegrity-Based Implant Technology Platform to strive for anatomical reduction of the fractured patella and stable fixation.

As is shown in FIG. 17, the Patella Tensegrity Device 1700 consists of multiple suture tunnel screws 1710 with a quick connect “T-lock” tensile element or suture locking mechanism on the screw heads.

Multiple sutures 1720 can be routed through each tunnel screw 1710 and then T-locked to the other screw heads to wrap and compress the fragmented patella in a tensegrity suture web. The sutures 1710 can be tension adjusted and locked, loosened, and relocked as needed.

The tensile element or suture locking system according to the present invention comais knotless, making it efficient and simple to use.

The advantages of the Patella Tensegrity Device are:
Fragment compression in all directions: The T-lock screw is a threaded differential pitch headless screw providing interfragmentary compression; the tensegrity suture web supplements and supersedes both the tension band device and cerclage loop to provide compression of the small, fragile fragments in all directions.
Mechanical advantage: multiple strands of heavy braided sutures strengthen fixation and decrease the chance of the implant cutting through the bone; more efficient system for routing the sutures compared to metal wires.
Lower risk of implant removal surgery: reduced implant protrusion and less soft tissue irritation from knotless T-lock suture anchor in screwheads compared to the conventional metal hardware; screws are headless and buried in bone; sutures are more flexible and produces less irritation symptoms in the long term.
Minimally invasive and simplified surgical procedure: no need to bend/cut K-wires; suture tensioning is performed using easy-to-use instruments and can be retightened if needed (vs. hand tied knots); minimal damage to soft tissue and extensor mechanism; reduced surgery time.

### Example 3: Tensegrity Device for Distal Humerus Ligament Reconstruction and Stability (Elbow)

The elbow is a hinged joint made up of the humerus, ulna, and radius bones (Error! Reference source not found.). The ends of the bones are covered with cartilage that allows the joints to slide easily against one another and absorb shock. The bones are held together with ligaments that form the joint capsule, a fluid filled sac that surrounds and lubricates the joint.

The important ligaments of the elbow are the medial (ulnar) collateral ligament (MCL) and the lateral (radial) collateral ligament (LCL) on the inside and outside of the elbow, respectively. Together these ligaments provide the main source of stability for the elbow, holding the humerus and the ulna tightly together to form the hinge-like humeroulnar joint that allows the arm to bend and straighten and prevents dislocation.

A third ligament, the annular ligament, holds the radial head tight against the ulna. The important tendons of the elbow are the biceps tendon (attaches the biceps muscle on the front of the arm to the radius) and the triceps tendon (attaches the triceps muscle on the back of the arm to the ulna).

Elbow instability is a looseness in the elbow joint that may cause the joint to lock, catch, pop, or slide out of place during certain arm movements and often occurs as a result of trauma or sports injury such as elbow dislocation. This type of injury can damage the bone and ligaments that surround the elbow joint and work to keep it stable, and can lead to recurrent or chronic elbow instability.
Efficient procedure for passing suture brace-reinforced grafts through the bone in the desired location and direction using the T-lock tunnel screws and easy-to-use instruments. Better mechanical strength compared to graft/single suture anchors.
Double bundle configuration on both sides to supplement both bundles of the LCL and of the MCL for improved stability.
Only 3 T-lock screws are needed in total for 4 bundles. Minimizes the risk of iatrogenic fracture.
Long screw lengths in humerus and ulna (through both cortices) to provide maximal interference anchorage for a graft when needed.
Adjustable tensioning and secure anchoring of suture brace-reinforced grafts to the bone using the quick connect T-lock screw system.

FIG. 18a to FIG. 18i show the surgical technique to treat elbow instability by using the implant device according to the present invention.

The first step of the surgical process is to position the bone engagement elements 1820, 1830 at the elbow joint 1800 such that the bone engagement elements 1820, 1830 are engaged with the at least two pieces of bones at the elbow joint 1800. In treating elbow stability, the bone engagement elements 1820, 1830 may be an anchor screw.

Referring to FIG. 18a, a posterolateral incision 1810 is made in order to visualize reduction of joint and oppose ruptured ligaments. A large 4.0 mm anchor screw 1820 is placed at the humeral isometric point 1800, two 3.0 mm anchor screws 1830 are placed with both ends corresponding to lateral side (LUCL) and medial (MCL) insertions.

Referring to FIG. 18b, a further Medial incision 1815 is made in order to visualize medial side of joint, the ulna nerve is avoided posterior to the isometric point. A large I-lock threaded wire 1840 is introduced into the humeral anchor screw 1820. The suture 1850 is led by the T-lock threaded wire 1840 to pass through the passageway of the humeral anchor screw 1820 such that the suture 1850 is brought through the humeral anchor screw 1820 as is shown in FIG. 18c.

Referring to FIG. 18c, the large T-lock wire 1840 brings the heavy braided suture-braces 1850 through the humeral isometric point 1800. The heavy braided suture-braces 1850 have two ends 1851 and 1852 which are connected to two smaller wires 1841, 1842.

The two ends 1851 and 1852 of the heavy braided suture-braces 1850 are then led by the two wires 1841, 1842 to pass through the two smaller anchor screws 1830. As is shown in FIG. 18d, suture-brace corresponding to thread A 1851 is introduced through the first 3.0 mm T-lock anchor screw 1831.

Similarly, as is shown in FIG. 18e, the second suture-brace (thread B) 1852 is introduced through the other 3.0 mm T-lock anchor screw 1832 at the ulna.

Referring to FIG. 18f, the passing of the two threads 1851, 1852 through the two smaller anchor screws 1830 can be operated by a surgeon at the medial incision 1815, and thus reinforces the Anterio-medial bundle of the ulna collateral ligament and the Sublime-tubercular bundle.

Afterwards, all ends of the suture-brace 1850 is passed through the T-lock anchoring tool 1860 as is shown in FIG. 18g, which then hold the ends of the suture-brace 1850 together and subsequently fasten them to one of the bone engagement elements 1820, with a suitable amount of tension.

Referring to FIG. 18h, the end of the sutures 1850 are fastened to the humerus 4.0 mm T-lock screw 1820. The tension of the sutures 1850 with respect to the bone engagement elements 1820, 1830 can manually maintained by gently turning the knob located at the end of the T-lock anchoring tool 1860.

By passing the sutures 1850 through the passageways of the large 4.0 mm anchor screw 1820 and the two 3.0 mm anchor screw 1830, the bones of the elbow joint which are engaged with the bone engagement elements 1820, 1830 are affixed and held against each other stably by the tensile stress in the suture material 1850, and as such provide bone fixation to the unstable elbow joint 1800.

FIG. 18i shows the bone fixation system of the present invention which has been successfully deployed with respect to the bones of the elbow joint 1800. The bone engagement elements 1820, 1830, which are engaging with the bones of the elbow joint 1800, are held securely against each other by the tension of the suture material 1850. Therefore, the elbow joint is now stabilised with double-bundle suture-braces both at the medial (MCL) and the lateral side (LUCL).

Any remaining tissue remnant of the ligaments of a graft can be imbricated together with the T-lock suture braces. Early mobilization is recommended after this minimally invasive procedure.

1. A bone engagement element for engagement with bone of a subject and for releasable engagement with a tensile element, said bone engagement element having a longitudinal central axis and comprising:
body portion for engagement with bone tissue of a subject, said body portion having a distal end and a proximal end, wherein the body portion includes at least one engagement surface towards said proximal end and facing in a direction of at least from the distal end towards the proximal end;
a bush member engageable with the proximal end of said body portion and having an aperture extending therethrough and being co-axial with said longitudinal central axis, said bush member having at least one complimentary engagement surface and facing in a direction of from at least the distal end towards the proximal end and said at least one complimentary engagement surface being complimentary to said at least one engagement surface of the body portion; and
a fastener member threadedly engageable with the proximal end of said body portion, wherein the fastener member extends through the aperture of the bush member and being co-axial with said longitudinal central axis;
wherein upon the fastener member being advanced in a direction of from the proximal end towards the distal end of the body portion, the fastener member urges the bush member in a direction of from the proximal end towards the distal end of the body portion;
wherein upon the bush member being urged in a direction of from the proximal end towards the distal end of the body portion, the at least one complimentary engagement surface of the bush member is urged towards said at least one engagement surface of the body portion to lock any elongate element disposed between the at least one engagement surface and the at least one complimentary engagement surface.

2. A bone engagement element according to claim 1, wherein the bone engagement element is a bone screw.

3. A bone engagement element according to claim 2, wherein the bone screw is a cortical bone screw or cancellous bone screw.

3. A bone engagement element according to claim 1, wherein the bone engagement element is a locking screw.

4. A bone engagement element according to claim 3, wherein the locking screw is a for engagement with a threaded aperture in an intermedullary fixation device.

5. A bone engagement element according to any one of the preceding claims, wherein the fastener member includes a threaded portion for threaded engagement with the proximal portion of the body portion, and an engagement portion for receiving for receiving an external drive element to allow for rotation of the fastener member about its longitudinal axis.

6. A bone engagement element according to any one of the preceding claims, where in the bush member is axially movable relative to the longitudinal axis of the body portion, and wearing the bush member is restricted from rotational movement relative to the body portion about the longitudinal axis of the body portion.

7. A bone engagement element according to any one of the preceding claims, wherein the bush member and the body portion are engaged by way of a key cooperate structure which prevents rotational motion of the bush member relative to the Longitudinal axis of the body portion.

8. A bone engagement element according to any one of the preceding claims, wherein said engagement surface is inclined radially outwardly and proximally so as to provide an outwardly extending inclined engagement surface.

9. A bone engagement element according to any one of the preceding claims, wherein the complementary engagement surface is distantly facing and inclined in a direction of outwardly radially and proximately.

10. A bone engagement element according to any one of the preceding claims, where in the engagement surface and the complimentary engagement surface are radially outwardly offset from the longitudinal central axis.

11. A bone engagement element according to any one of the preceding claims, where in a recess is provided at the proximal end portion of the body portion for receiving the bush member, and there is further provided one or more channels through which a tensile element may extend.

12. A bone engagement element according to claim 11, wherein said one or more channels channel is provided as one or more passageways.

13. A fixation system for the fixation of a first bone fragment relative to a second bone fragment; said system comprising;
a plurality of bone engagement elements according to any one of the preceding claims; and
one or more tensile members for engagement with said plurality of bone engagement elements;
wherein upon said tensile members engaged with said plurality of bone engagement elements and said bone engagement elements engaged with two or more bone fragments, said two or more bone fragments rigidly affixed relative to each other from tensile stress in said tensile member.

14. A fixation system for the fixation of a first bone fragment relative to a second bone fragment; said system comprising:
at least one first bone engagement element engagable with a first bone fragment, wherein said bone engagement element has an elongate body portion, a proximal end, a distal end, and a passageway extending through the body portion from the proximal end to the distal end,
at least one further bone engagement element engageable with a further bone fragment and having a tensile element securement portion for affixing a tensile element to said at least one further bone engagement element;
wherein upon tensile element passing the least one first bone engagement element when engaged with a first bone fragment is fixed relative to the at least one further bone engagement element when engaged with a first bone fragment by said tensile element securement portion; such that the first bone fragment is affixed relative to the further bone fragment from tensile stress in said tensile element material.

15. A fixation system according to claim 14, wherein the least one first bone engagement element includes a tensile element securement portion for affixing said tensile element to said least one first bone engagement element at the proximal end.

16. A fixation system according to claim 14 or claim 15, wherein the least one first bone engagement element includes a tensile element securement portion for affixing said tensile element to said least one first bone engagement element at the proximal end.

17. A fixation system according to claim 14, wherein the least one first bone engagement element includes a tensile element portion for affixing said tensile element to said least one first bone engagement element at the proximal end and the distal end.

18. A fixation system according to any one of claims 14 to 17, wherein the at least one further bone engagement element includes a suture securement portion for affixing said tensile element to said least one first bone engagement element at the proximal end.

19. A fixation system according to any one of claims 14 to 18, wherein the at least one further bone engagement element includes a tensile element securement portion for affixing said tensile element to said least one first bone engagement element at the proximal end.

20. A fixation system according to any one of claims 14 to 18, wherein the at least one further bone engagement element includes a tensile element securement portion for affixing said to said least one first bone engagement element at the proximal end and the distal end.

21. A fixation system according to any one of claims 14 to 20, wherein the at least a first bone engagement element is a bone screw.

22. A fixation system according to claim 21, wherein the bone screw is a bi-cortical bone screw.

23. A fixation system according to any one of claims 14 to 22, wherein the at least a further bone engagement element is a bone screw.

24. A fixation system according to claim 23, wherein the bone screw is a bi-cortical bone screw.

25. A fixation system according to any one of claims 14 to 24, wherein the system provides a three-dimensional structural support arrangement, which opposes relative movement between the first bone fragment and the further bone fragment.

26. A method of securing a plurality of bone fragments relative to each other for fracture healing and union, said method includes the steps of
(i) engaging a plurality of bone engagement elements with such bone fragments, wherein said bone engagement element has an elongate body portion, a proximal end, a distal end, and a passageway extending through the body portion from the proximal end to the distal end and having a tensile element securement portion for affixing a tensile element thereto
(ii) affixing a tensile element to a first bone engagement element of plurality of bone engagement elements which is engaged with a first bone fragment of said plurality of bone fragments; and
(ii) tensioning said tensile element and engaging said tensile element with a further bone engagement element which is affixed to a further bone fragment of said plurality of bone fragments;
wherein upon tensile element passing the least one first bone engagement element when engaged with a first bone fragment is fixed relative to the at least one further bone engagement element when engaged with a first bone fragment by said tensile element securement portion; such that the first bone fragment is affixed relative to the further bone fragment from tensile stress in said tensile element material.

27. A kit for securing a plurality of bone fragments relative to each other for fracture healing and union; said kit comprising:
plurality of bone engagement elements according to any one of claims 1 to 12; and
one or more tensile elements
